(19) United States
(12) Patent Application Publication
(10) Pub. No.: US 2025/0009726 A1
(43) Pub. Date: Jan. 9, 2025

(54) A PHARMACEUTICAL COMPOSITION OF FXR AGONIST AND ITS PREPARATION METHOD

(71) Applicant: GANNEX PHARMA CO., LTD., Shanghai (CN)

(72) Inventors: Kunhua DONG, Shanghai (CN); Jinzi Jason WU, Shanghai (CN)

(21) Appl. No.: 18/691,006

(22) PCT Filed: Sep. 9, 2021
(86) PCT No.: PCT/CN2021/117440

§ 371 Date: Mar. 11, 2024

(51) Int. Cl. A61K 31/4439; A61K 9/20; A61K 47/32

(52) U.S. Cl. CPC A61K 31/4439; A61K 9/2018; A61K 9/2054; A61K 9/2095; A61K 47/32

(57) ABSTRACT

A pharmaceutical composition comprises, in weight parts, the following components: (a) 1 part compound as shown in formula (I) (b) 0.2 to 15 parts polyvinylpyrrolidone; and (c) 1 part to 25 parts other pharmaceutically acceptable excipients. This pharmaceutical composition can be used for the treatment of steatohepatitis.

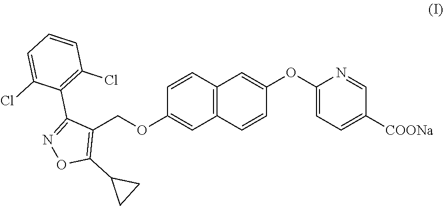

### FIELD

The present application generally relates to pharmaceutical formulations, and in particular to pharmaceutical formulations for medical treatments of liver diseases, such as non-alcoholic fatty liver disease (NAFLD) and non-alcoholic steatohepatitis (NASH).

### BACKGROUND

Nonalcoholic fatty liver disease (NAFLD) is the build up of extra fat in liver cells that is not caused by alcohol. It is normal for the liver to contain some fat. However, if more than 5%-10% percent of the liver's weight is fat, then it is called a fatty liver (steatosis). Nonalcoholic fatty liver disease (NAFLD) can be classified histologically into nonalcoholic fatty liver or nonalcoholic steatohepatitis (NASH). The worldwide prevalence of NAFLD is around 25%, and that of NASH ranges from 1.5% to 6.45%. Nonalcoholic fatty liver disease (NAFLD) is a clinicopathological term that encompasses a disease spectrum ranging from simple triglyceride accumulation in hepatocytes to hepatic steatosis with inflammation (nonalcoholic steatohepatitis, NASH) to fibrosis and cirrhosis. Hepatic insulin resistance is associated with steatosis.

The more severe form of NAFLD is called nonalcoholic steatohepatitis (NASH). NASH causes the liver to swell and become damaged. An increase in liver triglycerides can lead to increased oxidative stress in the hepatocytes, and the progression of hepatic steatosis to NASH. Oxidative stress results from an imbalance between pro-oxidant and antioxidant chemical species that leads to oxidative damage. Oxidation of fatty acids is an important source of reactive oxygen species (ROS). Some of the consequences of increased ROS is depleted ATP, destruction of membranes via lipid peroxidation, and release of proinflammatory cytokines. An increase in liver triglycerides may lead to increased oxidative stress in the hepatocytes, and the progression of hepatic steatosis to NASH. Human livers with NASH have increased lipid peroxidation and impaired mitochondrial function. This can result in cell death, hepatic stellate cell activation and fibrosis and inflammation. All of these activities may cause patients with NAFLD to be at risk for NASH, a more serious disease with higher risk of liver cirrhosis and hepatocellular carcinoma.

There is a continued need for effective treatments of NAFLD, and in particular NASH. The formulations described herein address this need.

### SUMMARY

One aspect of the present application relates to a pharmaceutical composition comprising:
(a) a compound of formula (I)

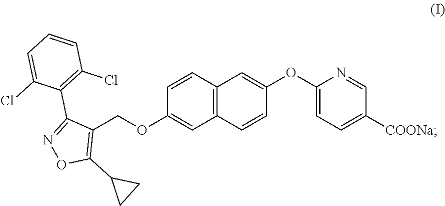

and
(b) one or more solubilizing agents.

In some embodiments, the pharmaceutical composition further comprises (c) one or more other pharmaceutically acceptable excipients.

In some embodiments, the pharmaceutical composition comprises, in weight parts:
1 part of compound of formula (I);
0.2 to 15 parts of one or more solubilizing agents; and
1 to 25 parts of one or more other pharmaceutically acceptable excipients.

In some embodiments, the one or more solubilizing agents comprise polyvinylpyrrolidone. In some embodiments, the one or more solubilizing agents consists of polyvinylpyrrolidone.

In some embodiments, the pharmaceutical composition comprises, in weight parts, 1 part of the compound of formula (I); 1 to 10 parts of polyvinylpyrrolidone; and 1.7 to 20 parts of one or more other pharmaceutically acceptable excipients.

In some embodiments, the one or more other pharmaceutically acceptable excipients are selected from the group consisting of fillers, disintegrants, lubricants and glidants.

In some embodiments, the pharmaceutical composition comprises one or more fillers selected from the group consisting of lactose, mannitol, silicified microcrystalline cellulose, dibasic calcium phosphate, microcrystalline cellulose, starch and pregelatinized starch. In related embodiments, the one or more fillers comprise mannitol and/or silicified microcrystalline cellulose.

In some embodiments, the one or more fillers comprise mannitol and silicified microcrystalline cellulose, and the pharmaceutical composition comprises polyvinylpyrrolidone and the one or more fillers at a polyvinylpyrrolidone-to-filler weight ratio of 1:0.3 to 1:7, preferably 1:1 to 1:2, and more preferably 1:1.2 to 1:1.6. In some embodiments, the pharmaceutical composition comprises polyvinylpyrrolidone and the one or more fillers at a polyvinylpyrrolidone-to-filler weight ratio of 1:1.4.

In some embodiments, the pharmaceutical composition comprises one or more disintegrants selected from of the group consisting of cross-linked polyvidone, croscarmellose sodium and croscarmellose sodium.

In some embodiments, the one or more disintegrant comprises cross-linked polyvidone. In some embodiments, the pharmaceutical composition comprises polyvinylpyrrolidone and cross-linked polyvidone at a polyvinylpyrrolidone-to-cross-linked polyvidone weight ratio of 1:0.1 to 1:2, preferably 1:0.2 to 1:1, and more preferably 1:0.3 to 1:0.5. In some embodiments, the pharmaceutical composition comprises polyvinylpyrrolidone and cross-linked polyvidone at a polyvinylpyrrolidone-to-cross-linked polyvidone weight ratio of 1:0.4.

In some embodiments, the pharmaceutical composition comprises one or more lubricants selected from of the group consisting of sodium stearyl fumarate, magnesium stearate and talc.

In some embodiments, the one or more lubricant comprises sodium stearyl fumarate. In some embodiments, the pharmaceutical composition comprises polyvinylpyrrolidone and sodium stearyl fumarate at a polyvinylpyrrolidone-to-sodium stearyl fumarate weight ratio of 1:0.04 to 1:1, preferably 1:0.1 to 1:0.8, and more preferably 1:0.1 to 1:0.3. In some embodiments, the pharmaceutical composition comprises polyvinylpyrrolidone and sodium stearyl fumarate at a polyvinylpyrrolidone-to-sodium stearyl fumarate weight ratio of 1:0.2.

In some embodiments, the pharmaceutical composition comprises one or more glidants selected from one or more of colloidal silicon dioxide, magnesium aluminum silicate and polyethylene glycol.

In some embodiments, the one or more glidants comprise colloidal silicon dioxide. In some embodiments, the pharmaceutical composition comprises polyvinylpyrrolidone and colloidal silicon dioxide at a polyvinylpyrrolidone-to-colloidal silicon dioxide weight ratio of 1:0.01 to 1:0.2, preferably 1:0.22 to 1:0.1, and more preferably 1:0.04 to 1:0.05.

In some embodiments, the compound of formula (I) is present in the pharmaceutical composition in an amorphous form.

In some embodiments, the compound of formula (I) is present in the pharmaceutical composition in the form of particles with particle sizes of D₅₀≤22.3 μm and D₉₀≤68.0 μm.

In some embodiments, the pharmaceutical composition is in a solid dosage form.

In some embodiments, the pharmaceutical composition is in the form of a tablet.

In some embodiments, the pharmaceutical composition comprises, in weight parts, 1 part of the compound of formula (I); 1 part of polyvinylpyrrolidone; 0.8 part of mannitol; 0.6 part of silicified microcrystalline cellulose; 0.4 part of cross-linked polyvidone; and 0.2 part of sodium stearyl fumarate.

In some embodiments, the pharmaceutical composition comprises, in weight parts, 1 part of the compound of formula (I); 10 part of polyvinylpyrrolidone; 8 part of mannitol; 6 part of silicified microcrystalline cellulose; 4 part of cross-linked polyvidone; and 2 part of sodium stearyl fumarate.

Another aspect of the application is a method of making a pharmaceutical composition as described herein. In some embodiments, the method comprises the steps of: (1) mixing the compound of formula (I) with one or more solubilizing agents and/or one or more other pharmaceutically acceptable excipients to form a first mixture, (2) granulating the first mixture to form a granulation product; and (3) produce the pharmaceutical composition in a dosage form with the granulation product.

In some embodiments, the method comprises the steps of: (1) mixing the compound of formula (I) with one or more solubilizing agents and/or one or more other pharmaceutically acceptable excipients to form a granulation mixture, (2) granulating the granulation mixture to form a granulation product; (3) blending the granulation product with one or more additional pharmaceutically acceptable carrier to form a tableting mixture; and (4) compressing the tableting mixture into tablets.

In some embodiments, one or more of the compound of formula (I), the one or more solubilizing agents and/or the one or more other pharmaceutically acceptable excipients are grinded and/or sieved before the mixing step. In some embodiments, compound of formula (I) is grinded and/or sieved with a 30-mesh sieve before the mixing step, and the one or more solubilizing agents and/or the one or more other pharmaceutically acceptable carriers are grinded and/or sieved with a 40-mesh sieve before the mixing step. In some embodiments, one or more of the compound of formula (I), the one or more solubilizing agents and/or the one or more other pharmaceutically acceptable excipients are grinded by mechanical grinding/pulverization and/or airflow grinding/pulverization.

In some embodiments, the method comprises the steps of: (1) sieving the compound of formula (I), polyvinylpyrrolidone, and/or one or more other pharmaceutically acceptable excipients to form sieved products; (2) blending the sieved products desired weight ratios to form a granulation mixture; (3) subjecting the granulation mixture to a granulation process to form a granulation product; (4) sieving the granulation product; (5) mixing the sieved granulation product with one or more additional pharmaceutically acceptable excipients to form a tableting mixture; and (6) compressing the tableting mixture into dosage form tablets.

In some embodiments, the compounds of formula (I) is sieved with a 30-mesh sieve, the polyvinylpyrrolidone and the one or more other pharmaceutically acceptable excipients are sieved with a 40-mesh sieve, and the granulation product is sieved with a 20-mesh sieve.

In some embodiments, the one or more pharmaceutically acceptable excipients in step (2) are selected from the group consisting of the fillers, disintegrants, lubricants, and glidants.

In some embodiments, the method further comprises preferably, the weight ratio of the remaining filler to the filler used for granulation is 0:14 to 1:1.8, more preferably 0:14 to 0:1.4; preferably, the weight ratio of the remaining disintegrant to the disintegrant used for granulation is 1:2 to 22:1, preferably 1:2 to 1:1; preferably 1:1; preferably, the weight ratio of the remaining lubricant to the lubricant used for granulation is 1:1 to 1:9, preferably 1:6 to 1:9; preferably 1:9; preferably, the weight ratio of the remaining glidant to the glidant used for granulation is 1:1 to 1:1.375, preferably 1:1.

In certain embodiments, the method further comprises, in step (2), the compound shown in formula (I), polyvinylpyrrolidone as solubilizing agent, filler, and a part of disintegrant and lubricant are granulated in the weight portion, and then mixed with the remaining disintegrant and lubricant.

In certain embodiments, the method further comprises, preferably, the weight ratio of the remaining disintegrant to the disintegrant for granulation is 1:2 to 22:1, and preferably 1:2 to 1:1; preferably, the weight ratio of the remaining lubricant to the lubricant for granulation is 1:1 to 1:9, and preferably 1:6 to 1:9; and preferably 1:9.

Another aspect of the present application relates to a method of treating nonalcoholic steatohepatitis using the pharmaceutical composition of the present application.

An aspect of the application is a method of treating nonalcoholic steatohepatitis using a pharmaceutical composition made by the method described herein.

### BRIEF DESCRIPTION OF THE DRAWINGS

FIG. 1 shows the dissolution behavior of Formulation 1 and Formulation 4 tablets in pure water.

FIG. 2 shows the dissolution behavior of Formulation 1 and Formulation 4 tablets in phosphate buffer (pH 6.8).

FIG. 3 shows the dissolution behavior of Formulation 7 and Formulation 9 tablets at pH 6.8.

FIG. 4 shows the dissolution behavior of Formulation 7 and Formulation 9 tablets at pH 4.5.

FIG. 5 shows the dissolution behavior of Formulation 7 and Formulation 9 tablets at pH 1.0.

FIG. 6 shows the dissolution of tablets with different VA64 formulation amounts.

FIG. 7 shows the dissolution behavior of tablets with different meglumine formulation amounts.

FIG. 8 shows the dissolution behavior of Formulation 21 and Formulation 24 tablets.

FIG. 9 shows the dissolution behavior of Formulation 24 tablets (dissolution method with pH 1.0 followed by pH 6.8).

FIG. 10 shows the dissolution behavior of Formulation 24 tablets at pH 6.8.

FIG. 11 shows the dissolution behavior of Formulation 26 tablets (dissolution method with pH 1.0 followed by pH 6.8);

FIG. 12 showed the dissolution behavior of Formulation 26 tablets at pH 6.8;

FIG. 13 shows the dissolution of Formulation 24 and Formulation 27 tablets at pH 6.8;

FIG. 14 shows the dissolution of Formulation 24 and Formulation 27 tablets at pH 1.0 followed by pH 6.8.

While the present disclosure will now be described in detail, and it is done so in connection with the illustrative embodiments, it is not limited by the particular embodiments illustrated in the figures and the appended claims.

### DETAILED DESCRIPTION OF THE INVENTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

As used in this specification and the appended claims, the singular forms “a,” “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to “the value,” greater than or equal to the value” and possible ranges between values are also disclosed, as appropriately understood by the skilled artisan. For example, if the value “10” is disclosed the “less than or equal to 10” as well as “greater than or equal to 10” is also disclosed.

### I. Definitions

“Administering” means providing a pharmaceutical agent or composition to a subject, and includes, but is not limited to, administering by a medical professional and self-administering.

Administration of the compounds disclosed herein or the pharmaceutically acceptable salts thereof, or the additional therapeutic agents disclosed herein can be via any of the accepted modes of administration for agents that serve similar utilities including, but not limited to, orally, subcutaneously, intravenously, intranasally, topically, transdermally, intraperitoneally, intramuscularly, intrapulmonarily, vaginally, rectally, or intraocularly. Oral and parenteral administrations are customary in treating the indications that are the subject of the preferred embodiments.

“Parenteral administration,” means administration through injection or infusion. Parenteral administration includes, but is not limited to, subcutaneous administration, intravenous administration, intramuscular administration, intraarterial administration, and intracranial administration.

“In combination” or “combination” refers to the compound of formula (I) and at least one additional therapeutic agent being substantially effective in the body at a same time. Both can be administered substantially at the same time, or both can be administered at different times but have effect on the body at the same time. For example, “in combination” includes administering the compound of formula (I) before the administration of the at least one additional therapeutic agent, and subsequently administering the at least one additional therapeutic agent while functioning of the compound of formula (I) in the body is substantially extant. In addition, “in combination” includes administering the at least one additional therapeutic agent before the administration of the compound of formula (I), and subsequently administering the compound of formula (I) while functioning of the at least one additional therapeutic agent in the body is substantially extant. When a pharmaceutical composition is described as containing the compound of formula (I) and the at least one additional therapeutic agent in combination, this term refers to both agents being concurrently present in the composition. The terms “in combination” and “combination” may further relate to the advantageous use of the compound of formula (I) and the at least one additional therapeutic agent in the absence of concomitant treatment for liver diseases such as NAFLD or NASH.

“Active pharmaceutical ingredient” means the substance in a pharmaceutical composition that provides a desired therapeutic effect.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable excipient” or “pharmaceutically acceptable carrier” includes any and all solvents, diluents, emulsifiers, binders, buffers, dispersion media, coatings, antibacterial and antifungal agents, isotonic and absorption delaying agents and the like, or any other such compound as is known by those of skill in the art to be useful in preparing pharmaceutical formulations. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

A “unit dosage form” refers to a composition containing an amount of a compound that is suitable for administration to a subject, in a single dose, according to good medical practice. However, as further described below, the preparation of a single or unit dosage form however, does not imply that the dosage form is administered once per day or once per course of therapy.

A “loading dose” refers to an initial dose of a compound which is higher than subsequent doses.

A “maintenance dose” refers to a subsequent dose that follows a loading dose, and occurs later in time than a loading dose. One of ordinary skill in the art will be aware that the dosage form or mode of administration of a maintenance dose may be different from that used for the loading dose. In any of the embodiments disclosed herein, a maintenance dose may comprise administration of the unit dosage form on any dosing schedule contemplated herein, including but not limited to, monthly or multiple times per month, biweekly or multiple times each two weeks, weekly or multiple times per week, daily or multiple times per day. It is contemplated within the present disclosure that dosing holidays may be incorporated into the dosing period of the maintenance dose. Such dosing holidays may occur immediately after the administration of the loading dose or at any time during the period of administration of the maintenance dose. As used herein, the period of administration of the maintenance dose may be referred to as the “maintenance phase” of the treatment period.

The phrase, “mode of administration” refers to the means by which a compound is administered to a subject. As such, the phrase encompasses the dosage form (for example, a tablet, powder, dissolved liquid, suspension, emulsion, aerosol, etc.) and the mechanism by which the dosage form is applied to the subject (for example, by oral administration or injection). The “mode of administration” may further encompass the dose, dose amount, and dosing schedule by which a compound is administered to a subject. The phrase “duration of the treatment” refers to the time commencing with administration of the first dose and concluding with the administration of the final dose, such length of time being determined by one of ordinary skill in the art of treating a given disease.

The phrase “dosing holiday” refers to a period of 24 hours or more during which either no dose is administered to the subject, or a reduced dose is administered to the subject.

“Fatty liver diseases” and liver disorders include the primary fatty liver diseases, steatosis or nonalcoholic fatty liver (NAFL), non-alcoholic fatty liver disease (NAFLD), non-alcoholic steatohepatitis (NASH), and hepatocellular carcinoma (HCC). Fatty liver diseases are typically conditions wherein large vacuoles of triglyceride fat accumulate in liver cells via the process of steatosis (i.e., abnormal retention of lipids within a cell). Accumulation of fat may also be accompanied by a progressive inflammation of the liver (hepatitis), called steatohepatitis. By considering the contribution of alcohol, fatty liver disease may be termed alcoholic steatosis or non-alcoholic fatty liver disease (NAFLD).

“Nonalcoholic fatty liver disease (NAFLD)” is an umbrella term for a range of liver conditions affecting people who drink little to no alcohol. As the name implies, the main characteristic of NAFLD is too much fat stored in liver cells. NAFLD is increasingly common around the world, especially in Western nations. In the United States, it is the most common form of chronic liver disease, affecting about one-quarter of the population. Some individuals with NAFLD can develop “nonalcoholic steatohepatitis (NASH),” an aggressive form of fatty liver disease, which is marked by liver inflammation and may progress to advanced scarring (cirrhosis) and liver failure. This damage is similar to the damage caused by heavy alcohol use.

“Subject” as used herein, means a human or a non-human mammal, including but not limited to a dog, cat, horse, donkey, mule, cow, domestic buffalo, camel, llama, alpaca, bison, yak, goat, sheep, pig, elk, deer, domestic antelope, or a non-human primate selected for treatment or therapy.

A “subject suspected of having” means a subject exhibiting one or more clinical indicators of a disease or condition.

A “subject in need thereof” means a subject identified as in need of a therapy or treatment.

### II. Pharmaceutical Composition

One aspect of the present application relates to a pharmaceutical composition comprising a compound of formula (I):

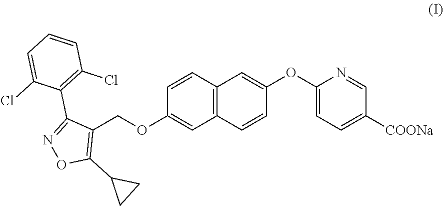

The compound shown in formula (I) (molecular formula of C₂₉H₁₉Cl₂N₂NaO₅, molecular weight 569.37 dalton, CAS No. 852948-13-1, chemical name 6-(6-(((5-cyclopropyl-3-(2,6-dichlorophenyl) isoxazol-4-yl) methoxy) naphthalene-2-yl) oxy) sodium nicotinate) is a farnesoid X receptor (FXR) agonist that is a drug candidate used for the treatment of nonalcoholic steatohepatitis.

The compound of formula (I) is a white to yellowish-brown solid in an amorphous form with pka=3.76 and Log D=5.76, which is practically insoluble in water, acetonitrile, tetrahydrofuran, ethyl acetate, and 0.01 mol/L sodium hydroxide solutions, and soluble in N, N-dimethylformamide, dimethyl sulfoxide, methanol. In addition, the solubility of this compound is pH-dependent, practically insoluble at low pH (pH=1.0-5.0) and very slightly soluble at pH=6.8. The solubility of drugs in the acidic environment of the gastrointestinal tract is a prerequisite for their transmembrane transport and absorption into the human body to exert efficacy. Therefore, the very low solubility of the compound limits its use for development as a drug candidate.

The technical problem to be solved by the present application is to provide a pharmaceutical composition that contains the compound of formula (I) and is suitable for industrial production. The technique described in the present application can maximize the dissolution of the compound of formula (I). The preparation process for the pharmaceutical composition is simple and easy, and without the use of organic solvents and high temperatures, thus ensuring the stability of the compound of formula (I) during the preparation process.

The present application discloses that the design of tablets with a specific formulation ratio of the compounds shown in formula (I) together with polyvinylpyrrolidone helped to greatly improve their in vitro solubility, while also ensuring the stability of the compounds shown in formula (I) due to the mild preparation conditions.

The advantages of the invention are as follows:
(1) the pharmaceutical composition of the invention can greatly improve the in vivo and in vitro dissolution rate of the compound shown in formula (I);
(2) the pharmaceutical composition of the compound shown in formula (I) can be prepared at a relatively mild temperature, avoiding the occurrence of drug degradation reaction under the high temperature process;
(3) the pharmaceutical composition process of the invention is simple and suitable for industrialized production, and can also effectively avoid the occurrence of dust pollution in the granulation process, explosion prevention and organic solvent residue in the spray drying solid dispersion process, and high temperature degradation during hot melt extrusion.

In some embodiments, the pharmaceutical composition comprises the compound of formula (I) and one or more solubilizing agents. Examples of the solubilizing agent include, but are not limited to, sodium lauryl sulfate, Poloxamer 188, Copovidone VA64. In some embodiments, the one or more solubilizing agents comprise polyvinylpyrrolidone. In some embodiments, the one or more solubilizing agents consists of polyvinylpyrrolidone.

In some embodiments, the pharmaceutical composition further comprises one or more other pharmaceutically acceptable excipients.

In some embodiments, the pharmaceutical composition comprises, in weight parts, 1 part of compound of formula (I), 0.2 to 15 parts of polyvinylpyrrolidone as solubilizing agent, and 1 to 25 parts of one or more other pharmaceutically acceptable excipients.

In some embodiments, the pharmaceutical composition comprises, in weight parts, 1 part of the compound of formula (I); 1 to 10 parts of polyvinylpyrrolidone; and 1.7 to 20 parts of one or more other pharmaceutically acceptable excipients.

In some embodiments, the one or more other pharmaceutically acceptable excipients are selected from the group consisting of fillers, disintegrants, lubricants and glidants.

In some embodiments, the pharmaceutical composition comprises one or more fillers selected from the group consisting of lactose, mannitol, silicified microcrystalline cellulose, dibasic calcium phosphate, microcrystalline cellulose, starch and pregelatinized starch. In related embodiments, the one or more fillers comprise mannitol and/or silicified microcrystalline cellulose.

In some embodiments, the one or more fillers comprise mannitol and silicified microcrystalline cellulose, and the pharmaceutical composition comprises polyvinylpyrrolidone and the one or more fillers at a polyvinylpyrrolidone-to-filler weight ratio of 1:0.3 to 1:7, preferably 1:1 to 1:2, and more preferably 1:1.2 to 1:1.6. In some embodiments, the pharmaceutical composition comprises polyvinylpyrrolidone and the one or more fillers at a polyvinylpyrrolidone-to-filler weight ratio of 1:1.4 to 1:6.88. In some embodiments, the pharmaceutical composition comprises polyvinylpyrrolidone and the one or more fillers at a polyvinylpyrrolidone-to-filler weight ratio of 1:1.4.

In some embodiments, the pharmaceutical composition comprises one or more disintegrants selected from of the group consisting of cross-linked polyvidone, croscarmellose sodium and croscarmellose sodium.

In some embodiments, the one or more disintegrant comprises cross-linked polyvidone. In some embodiments, the pharmaceutical composition comprises polyvinylpyrrolidone and cross-linked polyvidone at a polyvinylpyrrolidone-to-cross-linked polyvidone weight ratio of 1:0.1 to 1:2, preferably 1:0.2 to 1:1, and more preferably 1:0.3 to 1:0.5. In some embodiments, the pharmaceutical composition comprises polyvinylpyrrolidone and cross-linked polyvidone at a polyvinylpyrrolidone-to-cross-linked polyvidone weight ratio of 1:0.4.

In some embodiments, the pharmaceutical composition comprises one or more lubricants selected from of the group consisting of sodium stearyl fumarate, magnesium stearate and talc.

In some embodiments, the one or more lubricant comprises sodium stearyl fumarate. In some embodiments, the pharmaceutical composition comprises polyvinylpyrrolidone and sodium stearyl fumarate at a polyvinylpyrrolidone-to-sodium stearyl fumarate weight ratio of 1:0.04 to 1:1, preferably 1:0.1 to 1:0.8, and more preferably 1:0.1 to 1:0.3. In some embodiments, the pharmaceutical composition comprises polyvinylpyrrolidone and sodium stearyl fumarate at a polyvinylpyrrolidone-to-sodium stearyl fumarate weight ratio of 1:0.2.

In some embodiments, the pharmaceutical composition comprises one or more glidants selected from one or more of colloidal silicon dioxide, magnesium aluminum silicate and polyethylene glycol.

In some embodiments, the one or more glidants comprise colloidal silicon. In some embodiments, the pharmaceutical composition comprises polyvinylpyrrolidone and colloidal silicon at a polyvinylpyrrolidone-to-colloidal silicon weight ratio of 1:0.01 to 1:0.2, preferably 1:0.22 to 1:0.1, and more preferably 1:0.04 to 1:0.05.

In some embodiments, the compound of formula (I) is present in the pharmaceutical composition in an amorphous form.

In some embodiments, the compound of formula (I) is present in the pharmaceutical composition in the form of particles with particle sizes of D₅₀≤22.3 μm and D₉₀≤68.0 μm.

In some embodiments, the pharmaceutical composition is in a solid dosage form.

In some embodiments, the pharmaceutical composition is in the form of a tablet.

In some embodiments, the pharmaceutical composition comprises, in weight parts, 1 part of the compound of formula (I); 1 part of polyvinylpyrrolidone; 0.8 part of mannitol; 0.6 part of silicified microcrystalline cellulose; 0.4 part of cross-linked polyvidone; and 0.2 part of sodium stearyl fumarate.

In some embodiments, the pharmaceutical composition comprises, in weight parts, 1 part of the compound of formula (I); 10 part of polyvinylpyrrolidone; 8 part of mannitol; 6 part of silicified microcrystalline cellulose; 4 part of cross-linked polyvidone; and 2 part of sodium stearyl fumarate.

In some embodiments, the pharmaceutical composition of the present application is in a solid dosage form, and preferably in a tablet form.

Table 1 provides an exemplary list of solubilizing agent and other excipient suitable for the pharmaceutical composition of the present application.

TABLE 1
Function, dosage and maximum dosage of excipients for an embodiment
of the pharmaceutical composition of the present application

| | | | Maximum dosage |
|---|---|---|---|
| | | dosage | ( mg/ Unit |
| Name of excipient | Usage | (mg/tablet) | dose) Limitation * |
| Silicified microcrystalline | Filler | 30.0 | Oral tablet: 568.0 |
| celluloseSMCC90 | | | |
| Polyvinylpyrrolidone VA64 | Solubilizer | 50.0 | Oral tablet: 853.8 |
| MannitolM100 | Filler | 40.0 | Oral tablet: 971.36 |
| CrospovidoneXL-10 | Disintegrant | 20.0 | Oral tablet: 395.19 |
| Sodium Stearyl Fumarate | Lubricant | 10.0 | Oral tablet: 29.3 |

* FDA Inactive Ingredient Search for Approved Drug Products, Database Last Updated: Apr. 23, 2020 (http://www.accessdata.fda.gov/scripts/cder/iig/index.cfm)

### III. Routes, Regimen and Dosages of Administration

Administration of the active agents described herein may be achieved by modulating the dosing schedule such that subjects experience periodic partial or full reductions in dosing for fixed amounts of time, followed by a resumption of dosing.

The pharmaceutical preparations described herein are preferably provided in unit dosage form. As used herein, a “unit dosage form” is a composition containing an amount of a compound that is suitable for administration to a subject, in a single dose, according to good medical practice. The preparation of a single or unit dosage form however, does not imply that the dosage form is administered once per day or once per course of therapy. A unit dosage form may comprise a single daily dose or a fractional sub-dose wherein several unit dosage forms are to be administered over the course of a day in order to complete a daily dose. According to the present disclosure, a unit dosage form may be given more or less often than once daily, and may be administered more than once during a course of therapy. Such dosage forms may be administered in any manner consistent with their formulation, including orally, parenterally, and may be administered as an infusion over a period of time (e.g., from about 30 minutes to about 2-6 hours). While single administrations are specifically contemplated, the preparations administered according to the methods described herein may also be administered as a continuous infusion or via an implantable infusion pump.

In some embodiments, the unit dose for the compound of formula (I) is 1 mg, 2.5 mg, 5 mg, 7.5 mg, 10 mg, 12.5 mg, 15 mg, 20 mg, 30 mg, 40 mg, 45 mg, 60 mg, 75 mg, 80 mg, 100 mg, 125 mg or 150 mg.

In some embodiments, the compound of formula (I) and the one or more additional therapeutic agents are administered at dosages substantially the same as the dosages at which they are administered in the respective monotherapies. In some embodiments, the compound of formula (I) is administered at a dosage which is less than (e.g., less than 90%, less than 80%), less than 70%, less than 60%>, less than 50%, less than 40%, less than 30%>, less than 20%, or less than 10%>) its monotherapy dosage. In some embodiments, the one or more additional therapeutic agents are administered at a dosage which is less than (e.g., less than 90%, less than 80%, less than 70%, less than 60%, less than 50%, less than 40%, less than 30%, less than 20%, or less than 10%) its monotherapy dosage. In one aspect, both the compound of formula (I) and the at least one additional therapeutic agent (e.g., additional therapeutic agents described herein) are administered at a dosage which is less than (e.g., less than 90%, less than 80%, less than 70%, less than 60%, less than 50, less than 40%, less than 30%, less than 20%, or less than 10%) their respective monotherapy dosages.

The actual unit dose of the active compounds described herein depends on the specific compound, and on the condition to be treated. In some embodiments, the dose may be from about 0.01 mg/kg to about 120 mg/kg or more of body weight, from about 0.05 mg/kg or less to about 70 mg/kg, from about 0.1 mg/kg to about 50 mg/kg of body weight, from about 1.0 mg/kg to about 10 mg/kg of body weight, from about 5.0 mg/kg to about 10 mg/kg of body weight, or from about 10.0 mg/kg to about 20.0 mg/kg of body weight.

In some embodiments, the unit dose may be less than 100 mg/kg, 90 mg/kg, 80 mg/kg, 70 mg/kg, 60 mg/kg, 50 mg/kg, 40 mg/kg, 30 mg/kg, 25 mg/kg, 20 mg/kg, 10 mg/kg, 7.5 mg/kg, 6 mg/kg, 5 mg/kg, 4 mg/kg, 3 mg/kg, 2.5 mg/kg, 1 mg/kg, 0.5 mg/kg, 0.1 mg/kg, 0.05 mg/kg or 0.005 mg/kg of body weight. In some embodiments, the actual unit dose is 0.05, 0.07, 0.1, 0.3, 1.0, 3.0, 5.0, 10.0 or 25.0 mg/kg of body weight. Thus, for administration to a 70 kg person, the dosage range would be from about 0.1 mg to 70 mg, from about 1 mg to about 50 mg, from about 0.5 mg to about 10 mg, from about 1 mg to about 10 mg, from about 2.5 mg to about 30 mg, from about 35 mg or less to about 700 mg or more, from about 7 mg to about 600 mg, from about 10 mg to about 500 mg, from about 20 mg to about 300 mg, or from about 200 mg to about 2000 mg.

In some embodiments, the actual unit dose of the compound of formula (I) is 5 mg. In some embodiments the actual unit dose of the compound of formula (I) is 10 mg. In some embodiments the actual unit dose of the compound of formula (I) is 15 mg. In some embodiments the actual unit dose of the compound of formula (I) is 20 mg. In some embodiments the actual unit dose of the compound of formula (I) is 25 mg. In some embodiments, the actual unit dose of the compound of formula (I) is 30 mg. In some embodiments, the actual unit dose of the compound of formula (I) is 45 mg. In some embodiments, the actual unit dose of the compound of formula (I) is 60 mg. In some embodiments, the actual unit dose of the compound of formula (I) is 150 mg or less. In some embodiments, the actual unit dose of the compound of formula (I) is 100 mg or less. In some embodiments, the actual unit dose of the compound of formula (I) is 60 mg or less. In some embodiments, the actual unit dose of the compound of formula (I) is 60 mg or less. In some embodiments, the actual unit dose of the compound of formula (I) is 45 mg or less.

In some embodiments, the mode of administration comprises administering a loading dose of the compound of formula (I) followed by a maintenance dose. In some embodiments, the loading dose is 300 mg or less; 250 mg or less, 200 mg or less, 150 mg or less, 100 mg or less, 75 mg or less, or 60 mg or less, 45 mg or less or 30 mg or less. In some embodiments, the maintenance dose is 30 mg or less; 20 mg or less, 15 mg or less, 10 mg or less, 7.5 mg or less, 5 mg or less, 2 mg or less, or 1 mg or less.

In some embodiments, the loading dose is administered over a period of one day. In some embodiments the loading dose is administered over a period of 2 days. In some embodiments the loading dose is administered over a period of 3 days. In some embodiments the loading dose is administered over a period of 4 days. In some embodiments the loading dose is administered over a period of 5, 6 or 7 days. In some embodiments, the loading dose is administered over a period of 8-14 days or fewer. In some embodiments, the loading dose is administered over a period of 14 days.

In some embodiments, dosages are administered daily for between one and thirty days, followed by a dosing holiday lasting for between one and thirty days.

In some embodiments, during the dosing holiday, no dose is administered.

In further embodiments, the compound of formula (I) and its metabolites are allowed to clear completely from the subject's body prior to administration of the next dose.

In some other embodiments, during the dosing holiday, a dose less than the usual daily dose is administered.

In some further embodiments, an amount of the administered compound of formula (I) less than the therapeutically effective amount is allowed to remain within the subject during the dosing holiday.

In some further embodiments, an amount of the administered compound of formula (I) sufficient to maintain therapeutic levels in the affected tissues is allowed to remain within the subject.

In some embodiments, the maximum serum concentration of the compound of formula (I) during the dosing schedule is less than 120 ng/ml, less than 100 ng/ml, less than 90 ng/ml, less than 80 ng/ml, less than 70 ng/ml, less than 60 ng/ml, or less than 50 ng/ml.

In some embodiments, the minimum serum concentration of the compound of formula (I) during the dosing schedule is less than 10 ng/ml, less than 1 ng/ml, less than 0.1 ng/ml, less than 0.01 ng/ml, or less than 0.001 ng/ml.

In some embodiments, the level of the compound of formula (I) administered during the dosing schedule may be undetectable during some portion of the dosing holiday.

In some embodiments, the maximum serum concentration of the of the compound of formula (I) during the dosing schedule is higher during an initial phase of administration, and lower in subsequent phases.

In some embodiments, the maximum serum concentration of the compound of formula (I) during the initial (loading) phase of administration is less than 500 ng/ml, less than 400 ng/ml, less than 300 ng/ml, less than 200 ng/ml, less than 150 ng/ml, less than 120 ng/ml, less than 100 ng/ml, less than 90 ng/ml, less than 80 ng/ml, less than 70 ng/ml, less than 60 ng/ml, or less than 50 ng/ml.

In some such embodiments, the maximum serum concentration of the compound of formula (I) during the initial phase of administration is from 5 ng/ml to 250 ng/ml. In some embodiments, the maximum serum concentration of the compound of formula (I) during the subsequent (maintenance) phase of administration is less than 350 ng/ml, less than 200 ng/ml, less than 120 ng/ml, less than 100 ng/ml, less than 90 ng/ml, less than 80 ng/ml, less than 70 ng/ml, less than 60 ng/ml, or less than 50 ng/ml, less than 40 ng/ml, less than 35 ng/ml, or less than 10 ng/ml.

One of ordinary skill in the art will readily be aware of such methods as exist in the art for the monitoring of serum concentrations of pharmaceutical agents, and means of adjusting dosages of the compounds disclosed herein in order to achieve the desired serum concentrations. In some embodiments, the weekly dose to be administered is 600 mg or less. In some embodiments, the weekly dose is to be administered is 500 mg or less, 400 mg or less, 300 mg or less, 200 mg or less, 100 mg or less, 50 mg or less, 40 mg or less, 25 mg or less, 10 mg or less, or 5 mg or less, or within a range defined by any two of the foregoing.

According to the present application, the dosing schedule may be varied in order to attain the desired therapeutic effect. In particular, variations in the dosing schedule as described may be repeated throughout the duration of the treatment.

For example, in certain embodiments, the first dosage may be higher, lower, or the same as the dosages following the first dosage. In addition, a loading dose may precede the disclosed dosing regimen, and a dosing holiday may or may not follow the administration of the loading dose.

The methods described herein may utilize any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions include compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropies, surface-active agents, and encapsulating substances. Optional pharmaceutically-active materials may be included, which do not substantially interfere with the activity of the compound. The amount of carrier employed in conjunction with the compound is sufficient to provide a practical quantity of material for administration per unit dose of the compound. Techniques and compositions for making dosage forms useful in the methods described herein are described in e.g., Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

Other formulations useful for systemic delivery of the active agent(s) include sublingual, buccal and nasal dosage forms. Such formulations typically comprise one or more of soluble filler substances, such as sucrose, sorbitol and mannitol; and binders, such as acacia, microcrystalline cellulose, carboxymethyl cellulose and hydroxypropyl methyl cellulose. Glidants, lubricants, sweeteners, colorants, antioxidants and flavoring agents disclosed above may also be included.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, other useful vehicles used in the ophthalmic preparations disclosed herein may include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. Tonicity adjustors include, but are not limited to, salts, particularly sodium chloride, potassium chloride, mannitol and glycerin, or any other suitable ophthalmically acceptable tonicity adjustor.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7.

Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates, such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65287-332. Antimicrobial agents, including but not limited to phenylmercuric nitrate, thimerosal, benzethonium chloride, benzalkonium chloride, phenol, cresol, and chlorobutanol may also be included to achieve a bacteriostatic or fungistatic solution.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The compound of formula (I) and/or the one or more additional therapeutic agents according to the methods of the present application described herein may be administered by oral, intravenous, intraarterial, intestinal, rectal, vaginal, nasal, pulmonary, topical, intradermal, transdermal, transbuccal, translingual, sublingual, or opthalmic administration, or any combination thereof.

When the compound of formula (I) is administered in combination with the one or more additional therapeutic agents, the one or more pharmaceutical agents may be administered simultaneously or sequentially. In some embodiments, the compound of formula (I) and/or the one or more additional therapeutic agents are administered by co-administration. The term “co-administration,” as used hereinafter, refers to any one of the following: simultaneous administration, sequential administration, overlapping administration, concomitant administration, interval administration, continuous administration, contemporaneous administration or any combination thereof. In some such embodiments of the method, sequential co-administration is carried out in any order.

In some embodiments, the compound of formula (I) and/or the one or more additional therapeutic agents are administered every other day for the duration of the treatment. In other embodiments, the compound of formula (I) and/or the one or more additional therapeutic agents are administered on two out of every three days for the duration of the treatment. In still other embodiments the compound of formula (I) and/or the one or more additional therapeutic agents are administered two out of every four days for the duration of the treatment.

In some embodiments, the compound of formula (I) and/or the one or more additional therapeutic agents are administered daily for one day, followed by a two day dosing holiday. In some embodiments, the compound of formula (I) and/or the one or more additional therapeutic agents are administered daily for one day, followed by a two day dosing holiday. In some embodiments, the compound of formula (I) and/or the one or more additional therapeutic agents are administered daily for one day, followed by a three day dosing holiday. In some embodiments, the compound of formula (I) and/or the one or more additional therapeutic agents are administered daily for one day, followed by a four day dosing holiday. In some embodiments, the compound of formula (I) and/or the one or more additional therapeutic agents are administered daily for one day, followed by a five day dosing holiday. In some embodiments, the compound of formula (I) and/or the one or more additional therapeutic agents are administered daily for one day, followed by a six day dosing holiday. In some embodiments, the compound of formula (I) and/or the one or more additional therapeutic agents are administered daily for one day, followed by a seven day dosing holiday. In some embodiments, the compound of formula (I) and/or the one or more additional therapeutic agents are administered daily for one day, followed by an eight day dosing holiday. In some embodiments, the compound of formula (I) and/or the one or more additional therapeutic agents are administered daily for one day, followed by a nine day dosing holiday. In some embodiments, the compound of formula (I) and/or the one or more additional therapeutic agents are administered daily for one day, followed by a ten day dosing holiday. In some embodiments, the compound of formula (I) and/or the one or more additional therapeutic agents are administered daily for one day, followed by an eleven day dosing holiday. In some embodiments, the compound of formula (I) and/or the one or more additional therapeutic agents are administered daily for one day, followed by a twelve day dosing holiday. In some embodiments, the compound of formula (I) and/or the one or more additional therapeutic agents are administered daily for one day, followed by a thirteen day dosing holiday. In some embodiments, the compound of formula (I) and/or the one or more additional therapeutic agents are administered daily for one day, followed by a fourteen day dosing holiday.

In some embodiments, the compound of formula (I) and/or the one or more additional therapeutic agents are administered daily for two days, followed by a one day dosing holiday. In some embodiments, the compound of formula (I) and/or the one or more additional therapeutic agents are administered daily for two days, followed by a two day dosing holiday. In some embodiments, dosages are administered daily for two days, followed by a three day dosing holiday. In some embodiments, the compound of formula (I) and/or the one or more additional therapeutic agents are administered daily for two days, followed by a four day dosing holiday. In some embodiments, the compound of formula (I) and/or the one or more additional therapeutic agents are administered daily for two days, followed by a five day dosing holiday. In some embodiments, the compound of formula (I) and/or the one or more additional therapeutic agents are administered daily for two days, followed by a six day dosing holiday. In some embodiments, the compound of formula (I) and/or the one or more additional therapeutic agents are administered daily for two days, followed by a seven day dosing holiday. In some embodiments, dosages are administered daily for two days, followed by an eight day dosing holiday. In some embodiments, the compound of formula (I) and/or the one or more additional therapeutic agents are administered daily for two days, followed by a nine day dosing holiday. In some embodiments, the compound of formula (I) and/or the one or more additional therapeutic agents are administered daily for two days, followed by a ten day dosing holiday. In some embodiments, the compound of formula (I) and/or the one or more additional therapeutic agents are administered daily for two days, followed by an eleven day dosing holiday. In some embodiments, the compound of formula (I) and/or the one or more additional therapeutic agents are administered daily for two days, followed by a twelve day dosing holiday. In some embodiments, the compound of formula (I) and/or the one or more additional therapeutic agents are administered daily for two days, followed by a thirteen day dosing holiday. In some embodiments, the compound of formula (I) and/or the one or more additional therapeutic agents are administered daily for two days, followed by a fourteen day dosing holiday.

In some embodiments, the compound of formula (I) and/or the one or more additional therapeutic agents are administered daily for three days, followed by a one day dosing holiday. In some embodiments, the compound of formula (I) and/or the one or more additional therapeutic agents are administered daily for three days, followed by a two day dosing holiday. In some embodiments, the compound of formula (I) and/or the one or more additional therapeutic agents are administered daily for three days, followed by a three day dosing holiday. In some embodiments, the compound of formula (I) and/or the one or more additional therapeutic agents are administered daily for three days, followed by a four day dosing holiday. In some embodiments, the compound of formula (I) and/or the one or more additional therapeutic agents are administered daily for three days, followed by a five day dosing holiday. In some embodiments, the compound of formula (I) and/or the one or more additional therapeutic agents are administered daily for three days, followed by a six day dosing holiday. In some embodiments, the compound of formula (I) and/or the one or more additional therapeutic agents are administered daily for three days, followed by a seven day dosing holiday. In some embodiments, the compound of formula (I) and/or the one or more additional therapeutic agents are administered daily for three days, followed by an eight day dosing holiday. In some embodiments, the compound of formula (I) and/or the one or more additional therapeutic agents are administered daily for three days, followed by a nine day dosing holiday. In some embodiments, the compound of formula (I) and/or the one or more additional therapeutic agents are administered daily for three days, followed by a ten day dosing holiday. In some embodiments, the compound of formula (I) and/or the one or more additional therapeutic agents are administered daily for three days, followed by an eleven day dosing holiday. In some embodiments, the compound of formula (I) and/or the one or more additional therapeutic agents are administered daily for three days, followed by a twelve day dosing holiday. In some embodiments, the compound of formula (I) and/or the one or more additional therapeutic agents are administered daily for three days, followed by a thirteen day dosing holiday. In some embodiments, the compound of formula (I) and/or the one or more additional therapeutic agents are administered daily for three days, followed by a fourteen day dosing holiday.

In some embodiments, the compound of formula (I) and/or the one or more additional therapeutic agents are administered daily for four days, followed by a one day dosing holiday. In some embodiments, the compound of formula (I) and/or the one or more additional therapeutic agents are administered daily for four days, followed by a two day dosing holiday. In some embodiments, the compound of formula (I) and/or the one or more additional therapeutic agents are administered daily for four days, followed by a three day dosing holiday. In some embodiments, the compound of formula (I) and/or the one or more additional therapeutic agents are administered daily for four days, followed by a four day dosing holiday. In some embodiments, the compound of formula (I) and/or the one or more additional therapeutic agents are administered daily for four days, followed by a five day dosing holiday. In some embodiments, the compound of formula (I) and/or the one or more additional therapeutic agents are administered daily for four days, followed by a six day dosing holiday. In some embodiments, the compound of formula (I) and/or the one or more additional therapeutic agents are administered daily for four days, followed by a seven day dosing holiday. In some embodiments, the compound of formula (I) and/or the one or more additional therapeutic agents are administered daily for four days, followed by an eight day dosing holiday. In some embodiments, the compound of formula (I) and/or the one or more additional therapeutic agents are administered daily for four days, followed by a nine day dosing holiday. In some embodiments, the compound of formula (I) and/or the one or more additional therapeutic agents are administered daily for four days, followed by a ten day dosing holiday. In some embodiments, the compound of formula (I) and/or the one or more additional therapeutic agents are administered daily for four days, followed by an eleven day dosing holiday. In some embodiments, the compound of formula (I) and/or the one or more additional therapeutic agents are administered daily for four days, followed by a twelve day dosing holiday. In some embodiments, the compound of formula (I) and/or the one or more additional therapeutic agents are administered daily for four days, followed by a thirteen day dosing holiday. In some embodiments, the compound of formula (I) and/or the one or more additional therapeutic agents are administered daily for four days, followed by a fourteen day dosing holiday.

In some embodiments, the compound of formula (I) and/or the one or more additional therapeutic agents are administered daily for five days, followed by a one day dosing holiday. In some embodiments, the compound of formula (I) and/or the one or more additional therapeutic agents are administered daily for five days, followed by a two day dosing holiday. In some embodiments, the compound of formula (I) and/or the one or more additional therapeutic agents are administered daily for five days, followed by a three day dosing holiday. In some embodiments, the compound of formula (I) and/or the one or more additional therapeutic agents are administered daily for five days, followed by a four day dosing holiday. In some embodiments, the compound of formula (I) and/or the one or more additional therapeutic agents are administered daily for five days, followed by a five day dosing holiday. In some embodiments, the compound of formula (I) and/or the one or more additional therapeutic agents are administered daily for five days, followed by a six day dosing holiday. In some embodiments, the compound of formula (I) and/or the one or more additional therapeutic agents are administered daily for five days, followed by a seven day dosing holiday. In some embodiments, the compound of formula (I) and/or the one or more additional therapeutic agents are administered daily for five days, followed by an eight day dosing holiday. In some embodiments, the compound of formula (I) and/or the one or more additional therapeutic agents are administered daily for five days, followed by a nine day dosing holiday. In some embodiments, the compound of formula (I) and/or the one or more additional therapeutic agents are administered daily for five days, followed by a ten day dosing holiday. In some embodiments, the compound of formula (I) and/or the one or more additional therapeutic agents are administered daily for five days, followed by an eleven day dosing holiday. In some embodiments, the compound of formula (I) and/or the one or more additional therapeutic agents are administered daily for five days, followed by a twelve day dosing holiday. In some embodiments, the compound of formula (I) and/or the one or more additional therapeutic agents are administered daily for five days, followed by a thirteen day dosing holiday. In some embodiments, the compound of formula (I) and/or the one or more additional therapeutic agents are administered daily for five days, followed by a fourteen day dosing holiday.

In some embodiments, the compound of formula (I) and/or the one or more additional therapeutic agents are administered daily for six days, followed by a one day dosing holiday. In some embodiments, the compound of formula (I) and/or the one or more additional therapeutic agents are administered daily for six days, followed by a two day dosing holiday. In some embodiments, the compound of formula (I) and/or the one or more additional therapeutic agents are administered daily for six days, followed by a three day dosing holiday. In some embodiments, the compound of formula (I) and/or the one or more additional therapeutic agents are administered daily for six days, followed by a four day dosing holiday. In some embodiments, the compound of formula (I) and/or the one or more additional therapeutic agents are administered daily for six days, followed by a five day dosing holiday. In some embodiments, the compound of formula (I) and/or the one or more additional therapeutic agents are administered daily for six days, followed by a six day dosing holiday. In some embodiments, the compound of formula (I) and/or the one or more additional therapeutic agents are administered daily for six days, followed by a seven day dosing holiday. In some embodiments, the compound of formula (I) and/or the one or more additional therapeutic agents are administered daily for six days, followed by an eight day dosing holiday. In some embodiments, the compound of formula (I) and/or the one or more additional therapeutic agents are administered daily for six days, followed by a nine day dosing holiday. In some embodiments, the compound of formula (I) and/or the one or more additional therapeutic agents are administered daily for six days, followed by a ten day dosing holiday. In some embodiments, the compound of formula (I) and/or the one or more additional therapeutic agents are administered daily for six days, followed by an eleven day dosing holiday. In some embodiments, the compound of formula (I) and/or the one or more additional therapeutic agents are administered daily for six days, followed by a twelve day dosing holiday. In some embodiments, the compound of formula (I) and/or the one or more additional therapeutic agents are administered daily for six days, followed by a thirteen day dosing holiday. In some embodiments, the compound of formula (I) and/or the one or more additional therapeutic agents are administered daily for six days, followed by a fourteen day dosing holiday.

In some embodiments, the compound of formula (I) and/or the one or more additional therapeutic agents are administered daily for seven days, followed by a one day dosing holiday. In some embodiments, the compound of formula (I) and/or the one or more additional therapeutic agents are administered daily for seven days, followed by a two day dosing holiday. In some embodiments, the compound of formula (I) and/or the one or more additional therapeutic agents are administered daily for seven days, followed by a three day dosing holiday. In some embodiments, the compound of formula (I) and/or the one or more additional therapeutic agents are administered daily for seven days, followed by a four day dosing holiday. In some embodiments, the compound of formula (I) and/or the one or more additional therapeutic agents are administered daily for seven days, followed by a five day dosing holiday. In some embodiments, the compound of formula (I) and/or the one or more additional therapeutic agents are administered daily for seven days, followed by a six day dosing holiday. In some embodiments, the compound of formula (I) and/or the one or more additional therapeutic agents are administered daily for seven days, followed by a seven day dosing holiday. In some embodiments, the compound of formula (I) and/or the one or more additional therapeutic agents are administered daily for seven days, followed by an eight day dosing holiday. In some embodiments, the compound of formula (I) and/or the one or more additional therapeutic agents are administered daily for seven days, followed by a nine day dosing holiday. In some embodiments, the compound of formula (I) and/or the one or more additional therapeutic agents are administered daily for seven days, followed by a ten day dosing holiday. In some embodiments, the compound of formula (I) and/or the one or more additional therapeutic agents are administered daily for seven days, followed by an eleven day dosing holiday. In some embodiments, dosage the compound of formula (I) and/or the one or more additional therapeutic agents are administered daily for seven days, followed by a twelve day dosing holiday. In some embodiments, the compound of formula (I) and/or the one or more additional therapeutic agents are administered daily for seven days, followed by a thirteen day dosing holiday. In some embodiments, the compound of formula (I) and/or the one or more additional therapeutic agents are administered daily for seven days, followed by a fourteen day dosing holiday.

In some embodiments, the compound of formula (I) and/or the one or more additional therapeutic agents are administered daily for eight days, followed by a one day dosing holiday. In some embodiments, the compound of formula (I) and/or the one or more additional therapeutic agents are administered daily for eight days, followed by a two day dosing holiday. In some embodiments, the compound of formula (I) and/or the one or more additional therapeutic agents are administered daily for eight days, followed by a three day dosing holiday. In some embodiments, the compound of formula (I) and/or the one or more additional therapeutic agents are administered daily for eight days, followed by a four day dosing holiday. In some embodiments, the compound of formula (I) and/or the one or more additional therapeutic agents are administered daily for eight days, followed by a five day dosing holiday. In some embodiments, the compound of formula (I) and/or the one or more additional therapeutic agents are administered daily for eight days, followed by a six day dosing holiday. In some embodiments, the compound of formula (I) and/or the one or more additional therapeutic agents are administered daily for eight days, followed by a seven day dosing holiday. In some embodiments, the compound of formula (I) and/or the one or more additional therapeutic agents are administered daily for eight days, followed by an eight day dosing holiday. In some embodiments, the compound of formula (I) and/or the one or more additional therapeutic agents are administered daily for eight days, followed by a nine day dosing holiday. In some embodiments, the compound of formula (I) and/or the one or more additional therapeutic agents are administered daily for eight days, followed by a ten day dosing holiday. In some embodiments, the compound of formula (I) and/or the one or more additional therapeutic agents are administered daily for eight days, followed by an eleven day dosing holiday. In some embodiments, the compound of formula (I) and/or the one or more additional therapeutic agents are administered daily for eight days, followed by a twelve day dosing holiday. In some embodiments, the compound of formula (I) and/or the one or more additional therapeutic agents are administered daily for eight days, followed by a thirteen day dosing holiday. In some embodiments, the compound of formula (I) and/or the one or more additional therapeutic agents are administered daily for eight days, followed by a fourteen day dosing holiday.

In some embodiments, the compound of formula (I) and/or the one or more additional therapeutic agents are administered daily for nine days, followed by a one day dosing holiday. In some embodiments, the compound of formula (I) and/or the one or more additional therapeutic agents are administered daily for nine days, followed by a two day dosing holiday. In some embodiments, the compound of formula (I) and/or the one or more additional therapeutic agents are administered daily for nine days, followed by a three day dosing holiday. In some embodiments, the compound of formula (I) and/or the one or more additional therapeutic agents are administered daily for nine days, followed by a four day dosing holiday. In some embodiments, the compound of formula (I) and/or the one or more additional therapeutic agents are administered daily for nine days, followed by a five day dosing holiday. In some embodiments, the compound of formula (I) and/or the one or more additional therapeutic agents are administered daily for nine days, followed by a six day dosing holiday. In some embodiments, the compound of formula (I) and/or the one or more additional therapeutic agents are administered daily for nine days, followed by a seven day dosing holiday. In some embodiments, the compound of formula (I) and/or the one or more additional therapeutic agents are administered daily for nine days, followed by an eight day dosing holiday. In some embodiments, the compound of formula (I) and/or the one or more additional therapeutic agents are administered daily for nine days, followed by a nine day dosing holiday. In some embodiments, the compound of formula (I) and/or the one or more additional therapeutic agents are administered daily for nine days, followed by a ten day dosing holiday. In some embodiments, the compound of formula (I) and/or the one or more additional therapeutic agents are administered daily for nine days, followed by an eleven day dosing holiday. In some embodiments, the compound of formula (I) and/or the one or more additional therapeutic agents are administered daily for nine days, followed by a twelve day dosing holiday. In some embodiments, the compound of formula (I) and/or the one or more additional therapeutic agents are administered daily for nine days, followed by a thirteen day dosing holiday. In some embodiments, the compound of formula (I) and/or the one or more additional therapeutic agents are administered daily for nine days, followed by a fourteen day dosing holiday.

In some embodiments, the compound of formula (I) and/or the one or more additional therapeutic agents are administered daily for ten days, followed by a one day dosing holiday. In some embodiments, the compound of formula (I) and/or the one or more additional therapeutic agents are administered daily for ten days, followed by a two day dosing holiday. In some embodiments, the compound of formula (I) and/or the one or more additional therapeutic agents are administered daily for ten days, followed by a three day dosing holiday. In some embodiments, the compound of formula (I) and/or the one or more additional therapeutic agents are administered daily for ten days, followed by a four day dosing holiday. In some embodiments, the compound of formula (I) and/or the one or more additional therapeutic agents are administered daily for ten days, followed by a five day dosing holiday. In some embodiments, the compound of formula (I) and/or the one or more additional therapeutic agents are administered daily for ten days, followed by a six day dosing holiday. In some embodiments, the compound of formula (I) and/or the one or more additional therapeutic agents are administered daily for ten days, followed by a seven day dosing holiday. In some embodiments, the compound of formula (I) and/or the one or more additional therapeutic agents are administered daily for ten days, followed by an eight day dosing holiday. In some embodiments, the compound of formula (I) and/or the one or more additional therapeutic agents are administered daily for ten days, followed by a nine day dosing holiday. In some embodiments, the compound of formula (I) and/or the one or more additional therapeutic agents are administered daily for ten days, followed by a ten day dosing holiday. In some embodiments, the compound of formula (I) and/or the one or more additional therapeutic agents are administered daily for ten days, followed by an eleven day dosing holiday. In some embodiments, the compound of formula (I) and/or the one or more additional therapeutic agents are administered daily for ten days, followed by a twelve day dosing holiday. In some embodiments, the compound of formula (I) and/or the one or more additional therapeutic agents are administered daily for ten days, followed by a thirteen day dosing holiday. In some embodiments, the compound of formula (I) and/or the one or more additional therapeutic agents are administered daily for ten days, followed by a fourteen day dosing holiday.

In some embodiments, the compound of formula (I) and/or the one or more additional therapeutic agents are administered daily for eleven days, followed by a one day dosing holiday. In some embodiments, the compound of formula (I) and/or the one or more additional therapeutic agents are administered daily for eleven days, followed by a two day dosing holiday. In some embodiments, the compound of formula (I) and/or the one or more additional therapeutic agents are administered daily for eleven days, followed by a three day dosing holiday. In some embodiments, the compound of formula (I) and/or the one or more additional therapeutic agents are administered daily for eleven days, followed by a four day dosing holiday. In some embodiments, the compound of formula (I) and/or the one or more additional therapeutic agents are administered daily for eleven days, followed by a five day dosing holiday. In some embodiments, the compound of formula (I) and/or the one or more additional therapeutic agents are administered daily for eleven days, followed by a six day dosing holiday. In some embodiments, the compound of formula (I) and/or the one or more additional therapeutic agents are administered daily for eleven days, followed by a seven day dosing holiday. In some embodiments, the compound of formula (I) and/or the one or more additional therapeutic agents are administered daily for eleven days, followed by an eight day dosing holiday. In some embodiments, the compound of formula (I) and/or the one or more additional therapeutic agents are administered daily for eleven days, followed by a nine day dosing holiday. In some embodiments, the compound of formula (I) and/or the one or more additional therapeutic agents are administered daily for eleven days, followed by a ten day dosing holiday. In some embodiments, the compound of formula (I) and/or the one or more additional therapeutic agents are administered daily for eleven days, followed by an eleven day dosing holiday. In some embodiments, the compound of formula (I) and/or the one or more additional therapeutic agents are administered daily for eleven days, followed by a twelve day dosing holiday. In some embodiments, the compound of formula (I) and/or the one or more additional therapeutic agents are administered daily for eleven days, followed by a thirteen day dosing holiday. In some embodiments, the compound of formula (I) and/or the one or more additional therapeutic agents are administered daily for eleven days, followed by a fourteen day dosing holiday.

In some embodiments, the compound of formula (I) and/or the one or more additional therapeutic agents are administered daily for twelve days, followed by a one day dosing holiday. In some embodiments, the compound of formula (I) and/or the one or more additional therapeutic agents are administered daily for twelve days, followed by a two day dosing holiday. In some embodiments, the compound of formula (I) and/or the one or more additional therapeutic agents are administered daily for twelve days, followed by a three day dosing holiday. In some embodiments the compound of formula (I) and/or the one or more additional therapeutic agents are administered daily for twelve days, followed by a four day dosing holiday. In some embodiments, the compound of formula (I) and/or the one or more additional therapeutic agents are administered daily for twelve days, followed by a five day dosing holiday. In some embodiments, the compound of formula (I) and/or the one or more additional therapeutic agents are administered daily for twelve days, followed by a six day dosing holiday. In some embodiments, the compound of formula (I) and/or the one or more additional therapeutic agents are administered daily for twelve days, followed by a seven day dosing holiday. In some embodiments, the compound of formula (I) and/or the one or more additional therapeutic agents are administered daily for twelve days, followed by an eight day dosing holiday. In some embodiments, the compound of formula (I) and/or the one or more additional therapeutic agents are administered daily for twelve days, followed by a nine day dosing holiday. In some embodiments, the compound of formula (I) and/or the one or more additional therapeutic agents are administered daily for twelve days, followed by a ten day dosing holiday. In some embodiments, the compound of formula (I) and/or the one or more additional therapeutic agents are administered daily for twelve days, followed by an eleven day dosing holiday. In some embodiments, the compound of formula (I) and/or the one or more additional therapeutic agents are administered daily for twelve days, followed by a twelve day dosing holiday. In some embodiments, the compound of formula (I) and/or the one or more additional therapeutic agents are administered daily for twelve days, followed by a thirteen day dosing holiday. In some embodiments, the compound of formula (I) and/or the one or more additional therapeutic agents are administered daily for twelve days, followed by a fourteen day dosing holiday.

In some embodiments, the compound of formula (I) and/or the one or more additional therapeutic agents are administered daily for thirteen days, followed by a one day dosing holiday. In some embodiments, the compound of formula (I) and/or the one or more additional therapeutic agents are administered daily for thirteen days, followed by a two day dosing holiday. In some embodiments, the compound of formula (I) and/or the one or more additional therapeutic agents are administered daily for thirteen days, followed by a three day dosing holiday. In some embodiments, the compound of formula (I) and/or the one or more additional therapeutic agents are administered daily for thirteen days, followed by a four day dosing holiday. In some embodiments the compound of formula (I) and/or the one or more additional therapeutic agents are administered daily for thirteen days, followed by a five day dosing holiday. In some embodiments, the compound of formula (I) and/or the one or more additional therapeutic agents are administered daily for thirteen days, followed by a six day dosing holiday. In some embodiments, the compound of formula (I) and/or the one or more additional therapeutic agents are administered daily for thirteen days, followed by a seven day dosing holiday. In some embodiments, the compound of formula (I) and/or the one or more additional therapeutic agents are administered daily for thirteen days, followed by an eight day dosing holiday. In some embodiments, the compound of formula (I) and/or the one or more additional therapeutic agents are administered daily for thirteen days, followed by a nine day dosing holiday. In some embodiments, the compound of formula (I) and/or the one or more additional therapeutic agents are administered daily for thirteen days, followed by a ten day dosing holiday. In some embodiments, the compound of formula (I) and/or the one or more additional therapeutic agents are administered daily for thirteen days, followed by an eleven day dosing holiday. In some embodiments, the compound of formula (I) and/or the one or more additional therapeutic agents are administered daily for thirteen days, followed by a twelve day dosing holiday. In some embodiments, the compound of formula (I) and/or the one or more additional therapeutic agents are administered daily for thirteen days, followed by a thirteen day dosing holiday. In some embodiments, the compound of formula (I) and/or the one or more additional therapeutic agents are administered daily for thirteen days, followed by a fourteen day dosing holiday.

In some embodiments, the compound of formula (I) and/or the one or more additional therapeutic agents are administered daily for fourteen days, followed by a one day dosing holiday. In some embodiments, the compound of formula (I) and/or the one or more additional therapeutic agents are administered daily for fourteen days, followed by a two day dosing holiday. In some embodiments, the compound of formula (I) and/or the one or more additional therapeutic agents are administered daily for fourteen days, followed by a three day dosing holiday. In some embodiments the compound of formula (I) and/or the one or more additional therapeutic agents are administered daily for fourteen days, followed by a four day dosing holiday. In some embodiments the compound of formula (I) and/or the one or more additional therapeutic agents are administered daily for fourteen days, followed by a five day dosing holiday. In some embodiments, the compound of formula (I) and/or the one or more additional therapeutic agents are administered daily for fourteen days, followed by a six day dosing holiday. In some embodiments, the compound of formula (I) and/or the one or more additional therapeutic agents are administered daily for fourteen days, followed by a seven day dosing holiday. In some embodiments, the compound of formula (I) and/or the one or more additional therapeutic agents are administered daily for fourteen days, followed by an eight day dosing holiday. In some embodiments, the compound of formula (I) and/or the one or more additional therapeutic agents are administered daily for fourteen days, followed by a nine day dosing holiday. In some embodiments, the compound of formula (I) and/or the one or more additional therapeutic agents are administered daily for fourteen days, followed by a ten day dosing holiday. In some embodiments, the compound of formula (I) and/or the one or more additional therapeutic agents are administered daily for fourteen days, followed by an eleven day dosing holiday. In some embodiments, the compound of formula (I) and/or the one or more additional therapeutic agents are administered daily for fourteen days, followed by a twelve day dosing holiday. In some embodiments, the compound of formula (I) and/or the one or more additional therapeutic agents are administered daily for fourteen days, followed by a thirteen day dosing holiday. In some embodiments, the compound of formula (I) and/or the one or more additional therapeutic agents are administered daily for fourteen days, followed by a fourteen day dosing holiday.

In some embodiments, the compound of formula (I) and/or the one or more additional therapeutic agents are administered daily for thirty days followed by a thirty day dosing holiday. In some embodiments, the compound of formula (I) and/or the one or more additional therapeutic agents are administered daily for thirty days followed by a 25-30 day dosing holiday. In some embodiments, the compound of formula (I) and/or the one or more additional therapeutic agents are administered daily for thirty days followed by a 20-25 day dosing holiday. In some embodiments, the compound of formula (I) and/or the one or more additional therapeutic agents are administered daily for thirty days followed by a 15-20 day dosing holiday. In some embodiments, the compound of formula (I) and/or the one or more additional therapeutic agents are administered daily for thirty days followed by a 10-15 day dosing holiday. In some embodiments, the compound of formula (I) and/or the one or more additional therapeutic agents are administered daily for thirty days followed by a 5-10 day dosing holiday. In some embodiments, the compound of formula (I) and/or the one or more additional therapeutic agents are administered daily for thirty days followed by a 1-5 day dosing holiday.

In some embodiments, the compound of formula (I) and/or the one or more additional therapeutic agents are administered daily for 25-30 days followed by a thirty day dosing holiday. In some embodiments, the compound of formula (I) and/or the one or more additional therapeutic agents are administered daily for 25-30 days followed by a 25-30 day dosing holiday. In some embodiments, the compound of formula (I) and/or the one or more additional therapeutic agents are administered daily for 25-30 days followed by a 20-25 day dosing holiday. In some embodiments, the compound of formula (I) and/or the one or more additional therapeutic agents are administered daily for 25-30 days followed by a 15-20 day dosing holiday. In some embodiments, the compound of formula (I) and/or the one or more additional therapeutic agents s are administered daily for 25-30 days followed by a 10-15 dosing holiday. In some embodiments, the compound of formula (I) and/or the one or more additional therapeutic agents are administered daily for 25-30 days followed by a 5-10 day dosing holiday. In some embodiments, dosages are administered daily for 25-30 days followed by a 1-5 day dosing holiday.

In some embodiments, the compound of formula (I) and/or the one or more additional therapeutic agents are administered daily for 20-25 days followed by a thirty day dosing holiday. In some embodiments, the compound of formula (I) and/or the one or more additional therapeutic agents are administered daily for 20-25 days followed by a 25-30 day dosing holiday. In some embodiments, the compound of formula (I) and/or the one or more additional therapeutic agents are administered daily for 20-25 days followed by a 20-25 day dosing holiday. In some embodiments, the compound of formula (I) and/or the one or more additional therapeutic agents are administered daily for 20-25 days followed by a 15-20 day dosing holiday. In some embodiments, the compound of formula (I) and/or the one or more additional therapeutic agents are administered daily for 20-25 days followed by a 10-15 dosing holiday. In some embodiments, the compound of formula (I) and/or the one or more additional therapeutic agents are administered daily for 20-25 days followed by a 5-10 day dosing holiday. In some embodiments, the compound of formula (I) and/or the one or more additional therapeutic agents are administered daily for 20-25 days followed by a 1-5 day dosing holiday.

In some embodiments, the compound of formula (I) and/or the one or more additional therapeutic agents are administered daily for 15-20 days followed by a thirty day dosing holiday. In some embodiments, the compound of formula (I) and/or the one or more additional therapeutic agents are administered daily for 15-20 days followed by a 25-30 day dosing holiday. In some embodiments, the compound of formula (I) and/or the one or more additional therapeutic agents are administered daily for 15-20 days followed by a 20-25 day dosing holiday. In some embodiments, the compound of formula (I) and/or the one or more additional therapeutic agents are administered daily for 15-20 days followed by a 15-20 day dosing holiday. In some embodiments, the compound of formula (I) and/or the one or more additional therapeutic agents are administered daily for 15-20 days followed by a 10-15 day dosing holiday. In some embodiments, the compound of formula (I) and/or the one or more additional therapeutic agents are administered daily for 15-20 days followed by a 5-10 day dosing holiday. In some embodiments, the compound of formula (I) and/or the one or more additional therapeutic agents are administered daily for 15-20 days followed by a 1-5 day dosing holiday.

In any of the foregoing embodiments, the daily dosing may be administered in one dose administered once or day, or in two or more divided doses administered multiple times per day. For example, the compounds described herein may be administered once per day, twice per day, three times per day, or four times per day.

In some embodiments, the compound of formula (I) and the one or more additional therapeutic agents are administered in synergistically effective amount.

### IV. Method of Preparation

The invention also provides a preparation method for the pharmaceutical composition as shown, which comprises the following steps:
mixing a compound of formula (I)

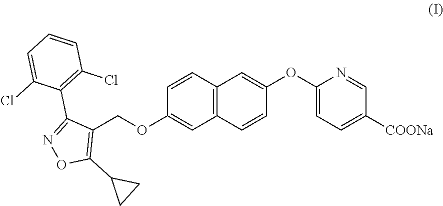

with one or more solubilizing agents and/or one or more other pharmaceutically acceptable excipients to form a first mixture;
granulating the first mixture to form a granulation product; and
producing the pharmaceutical composition in a dosage form with the granulation product.

In some embodiments, the producing step comprises blending the granulation product with one or more additional pharmaceutically acceptable carrier to form a tableting mixture; and compressing the tableting mixture into tablets.

In some embodiments, the method comprises the steps of:
(1) crushing/grinding and sieving the compound of formula (I), polyvinylpyrrolidone, and a first portion of other pharmaceutically acceptable excipients to form sieved products;
(2) mixing the sieved products to form a mixture;
(3) granulating the mixture to form a granulation product;
(4) mixing the granulation product with a second portion of other pharmaceutically acceptable excipients to form a tablet mixture; and
(5) compressing the tableting mixture into tablets.

In some embodiments, the crushing/grinding in step (1) is mechanical and/or airflow crushing/grinding. In some embodiments, the crushing/grinding in step (1) is mechanical grinding. In some embodiments, polyvinylpyrrolidone and other pharmaceutically acceptable excipients as solubilizing agents are sieved through a 40-mesh screen in step (1) and the compound of formula (I) are sieved through a 30-mesh screen in step 1.

Examples of the other pharmaceutically acceptable excipients include, but are not limited to, fillers, disintegrants, lubricants, and glidants.

In some embodiments, the other pharmaceutically acceptable excipients comprise a filler, and the weight ratio of (a) the filler in the first portion the other pharmaceutically acceptable excipients: (b) the filler in the second portion of the other pharmaceutically acceptable excipients is 0:14 to 1:1.8, preferably 0:14 to 0:1.4.

In some embodiments, the other pharmaceutically acceptable excipients comprise a disintegrant, and the weight ratio of (a) the disintegrant in the first portion the other pharmaceutically acceptable excipients: (b) the disintegrant in the second portion of the other pharmaceutically acceptable excipients is 1:2 to 22:1, preferably 1:2 to 1:1; preferably 1:1.

In some embodiments, the other pharmaceutically acceptable excipients comprise a lubricant, and the weight ratio of (a) the lubricant in the first portion the other pharmaceutically acceptable excipients: (b) the lubricant in the second portion of the other pharmaceutically acceptable excipients is 1:1 to 1:9, preferably 1:6 to 1:9; preferably 1:9.

In some embodiments, the other pharmaceutically acceptable excipients comprise a glidant, and the weight ratio of (a) the glidant in the first portion the other pharmaceutically acceptable excipients: (b) the glidant in the second portion of the other pharmaceutically acceptable excipients is 1:1 to 1:1.4, preferably 1:1.

In accordance with a specific embodiment of the present invention, the preparation method of the pharmaceutical composition comprises the following steps:
(1) pretreatment of the drug substance and excipients: passing the compound as shown in formula (I), polyvinylpyrrolidone, silicified microcrystalline cellulose, mannitol, cross-linked polyvidone, and sodium stearyl fumarate through a 40-mesh sieve, and passing the compound as shown in formula (I) through a 30-mesh or 40-mesh sieve;
(2) premixing: mixing the compound as shown in formula (I), polyvinylpyrrolidone, silicified microcrystalline cellulose, mannitol, and a portion of cross-linked polyvidone and sodium stearyl fumarate according to the weight;
(3) dry granulation: the mixture is dry granulated;
(4) total mixing: the remaining cross-linked polyvidone and sodium stearyl fumarate are mixed with granules obtained by dry granulation; and
(5) compression. Preferably, the weight ratio of the remaining added cross-linked polyvidone to the added cross-linked polyvidone for granulation is 1:1; the weight ratio of the remaining sodium stearyl fumarate added to the added sodium stearyl fumarate for granulation is 1:9.

The invention also provides the use of the drug preparation or the drug preparation prepared by the preparation method in preparing a drug for treating nonalcoholic steatohepatitis.

The present application is further illustrated by the following examples that should not be construed as limiting. The contents of all references, patents, and published patent applications cited throughout this application, as well as the Figures and Tables, are incorporated herein by reference.

### EXAMPLES

### Example 1. The Compatibility Test of API and Excipients

Mix the compound of formula (I) (hereinafter “COMPOUND I” or “API”) and excipients in proportion, and place them under the conditions of strong light (10° C., 4500Lx±500Lx), high temperature (60° C.) and high humidity (normal temperature, RH 92.5%) respectively for 17 days to investigate the compatibility of API and excipients. The mixing ratio of drug substance and excipients and the test results are shown in Table 2.

The results showed that: (1) When COMPOUND I drug substance and COMPOUND I drug substance were mixed with excipients, the appearance characteristics would change under high humidity conditions, which was caused by the easy moisture absorption of COMPOUND I drug substance. Under the light and high temperature conditions, there was no significant change in appearance. (2) COMPOUND I was stable under strong light, high temperature and high humidity conditions, and the total impurity fluctuations of related substances were less than 0.5%, without significant changes. (3) COMPOUND I drug substance was mixed with mannitol 200SD, silicified microcrystalline cellulose (trade name: PROSOLV) SMCC90, polyvinylpyrrolidone VA64, cross-linked polyvidone XL-10 and sodium stearyl fumarate in different proportions and placed at high temperature, high humidity and light for 5 days, 10 days and 17 days, respectively. The results showed that compared with 0 day, the total impurities fluctuated around 0.5%, no new impurities greater than 0.1% occurred, and the known impurities did not change significantly, indicating that COMPOUND I drug substance had good compatibility with various excipients.

TABLE 2
Compatibility test results of drug substance and excipients

| Type and | | | Related substances (%) | | | | | |
|---|---|---|---|---|---|---|---|---|
| proportion of | | | | | | | Other | |
| drug substance | | | COMPOUND | COMPOUND | COMPOUND | COMPOUND | maximum | Total |
| and excipients | Conditions | Appearance | I-B | I-IM1 | I-C | I-IM2 | impurities | impurities |
| COMPOUND I | 0 Day | Yellow | 0.2869 | 0.2264 | Not | Not | 0.1001 | 1.4056 |
| | | solid | | | detected | detected | | |
| | 60° C.-17 | Yellow | 0.2964 | 0.2278 | Not | Not | 0.1158 | 1.4686 |
| | Days | solid | | | detected | detected | | |
| | 25° C., | Yellow | 0.2896 | 0.2338 | Not | Not | 0.1135 | 1.4901 |
| | 4500Lx-17 | solid | | | detected | detected | | |
| | Days | | | | | | | |
| | normal | Yellowish | 0.2836 | 0.2318 | Not | Not | 0.1124 | 1.4400 |
| | temperatur, | brown | | | detected | detected | | |
| | 92.5% RH-17 | viscous | | | | | | |
| | Days | solid | | | | | | |
| COMPOUND I: | 0 Day | Off-white | 0.2753 | 0.2270 | Not | Not | 0.1051 | 1.3585 |
| Mannitol | | solid | | | detected | detected | | |
| 200SD = 1:5 | 60° C.-17 | Off-white | 0.2507 | 0.2338 | Not | Not | 0.1122 | 1.5838 |
| | Days | solid | | | detected | detected | | |
| | 25° C., | Off-white | 0.2125 | 0.2397 | Not | Not | 0.1196 | 1.6701 |
| | 4500Lx-17 | solid | | | detected | detected | | |
| | Days | | | | | | | |
| | normal | Off-white | 0.3120 | 0.2328 | Not | Not | 0.1208 | 1.4906 |
| | temperatur, | solid | | | detected | detected | | |
| | 92.5% RH-17 | | | | | | | |
| | Days | | | | | | | |
| COMPOUND I: | 0 Day | Off-white | 0.2745 | 0.2250 | Not | Not | 0.1147 | 1.1679 |
| Silicified | | solid | | | detected | detected | | |
| microcrystalline | 60° C.-17 | Off-white | 0.2479 | 0.2319 | Not | Not | 0.1135 | 1.3299 |
| cellu- | Days | solid | | | detected | detected | | |
| loseSMCC90 = 1:5 | 25° C., | Off-white | 0.2125 | 0.2413 | Not | Not | 0.1112 | 1.3288 |
| | 4500Lx-17 | solid | | | detected | detected | | |
| | Days | | | | | | | |
| | normal | Off-white | 0.2956 | 0.2321 | Not | Not | 0.1264 | 1.1255 |
| | temperatur, | solid | | | detected | detected | | |
| | 92.5% RH-17 | | | | | | | |
| | Days | | | | | | | |
| COMPOUND I: | 0 Day | Light | 0.2265 | 0.2264 | Not | Not | 0.1021 | 1.2978 |
| Polyvinylpyr- | | yellow | | | detected | detected | | |
| rolidoneVA64 = 1:1 | | solid | | | | | | |
| | 60° C.-17 | Light | 0.2405 | 0.2275 | Not | Not | 0.0971 | 1.4000 |
| | Days | yellow | | | detected | detected | | |
| | | solid | | | | | | |
| | 25° C., | Light | 0.2336 | 0.2302 | Not | Not | 0.1053 | 1.3492 |
| | 4500Lx-17 | yellow | | | detected | detected | | |
| | Days | solid | | | | | | |
| | normal | Yellowish | 0.2871 | 0.2315 | Not | Not | 0.0972 | 1.4225 |
| | temperatur, | brown | | | detected | detected | | |
| | 92.5% RH-17 | viscous | | | | | | |
| | Days | solid | | | | | | |
| COMPOUND I: | 0 Day | Yellow | 0.2827 | 0.2270 | Not | Not | 0.1060 | 1.3920 |
| CrospovidoneXL- | | solid | | | detected | detected | | |
| 10 = 20:1 | 60° C.-17 | Yellow | 0.2690 | 0.2306 | Not | Not | 0.1040 | 1.3648 |
| | Days | solid | | | detected | detected | | |
| | 25° C., | Yellow | 0.2443 | 0.2337 | Not | Not | 0.1064 | 1.4350 |
| | 4500Lx-17 | solid | | | | | | |
| | Days | | | | detected | detected | | |
| | normal | Yellowish | 0.2716 | 0.2294 | Not | Not | 0.0990 | 1.4162 |
| | temperatur, | brown | | | detected | detected | | |
| | 92.5% RH-17 | solid | | | | | | |
| | Days | | | | | | | |
| COMPOUND I: | 0 Day | Yellow | 0.2968 | 0.2310 | Not | Not | 0.1092 | 1.4511 |
| Sodium Stearyl | | solid | | | detected | detected | | |
| Fumarate = 20:1 | 60° C.-17 | Yellow | 0.2863 | 0.2262 | Not | Not | 0.1126 | 1.8083 |
| | Days | solid | | | detected | detected | | |
| | 25° C., | Yellow | 0.2563 | 0.2316 | Not | Not | 0.1130 | 1.4417 |
| | 4500Lx-17 | solid | | | detected | detected | | |
| | Days | | | | | | | |
| | normal | Yellowish | 0.2875 | 0.2272 | Not | Not | 0.1130 | 1.4090 |
| | temperatur, | brown | | | detected | detected | | |
| | 92.5% RH-17 | solid | | | | | | |
| | Days | | | | | | | |

### Example 2. Pre-Formulation Study

Studies on the crystallization process of COMPOUND I found only one type of amorphous powder without solvent.

Considering the limited number of API batches used in the COMPOUND I tablet study phase, the reference range for particle size control was tentatively limited to the mean±3σ (σ is the standard deviation) range of the three batches, i.e., D50≤22.3 μm, D90≤68.0 μm. The final control range for particle size was determined cumulatively based on stepwise scale-up of the drug substance and tablet processes, preparation of samples from pivotal clinical batches.

TABLE 3
Particle size and distribution of COMPOUND I API

| | Particle size and distribution | | | |
|---|---|---|---|---|
| | (mean ± SD, μm) | | | |
| Batch No. | MeanMv | D₁₀ | D₅₀ | D₉₀ |
| NASH1-23-00857-\textcircled{1} | 22.6 | 2.2 | 12.6 | 52.6 |
| COMPOUND I- | 11.1 | 0.9 | 3.0 | 32.1 |
| 20191230-1 | | | | |
| COMPOUND I- | 12.9 | 1.3 | 4.6 | 34.4 |
| 20200107-1 | | | | |
| COMPOUND I- | 18.2 | 2.2 | 11.4 | 42.0 |
| C20001 | | | | |
| σ (SD) | 5.2 | 0.7 | 4.8 | 9.2 |
| Mean | 16.2 | 1.7 | 7.9 | 40.3 |
| Mean-3σ | 0.5 | −0.3 | −6.5 | 12.5 |
| Mean + 3σ | 31.9 | 3.6 | 22.3 | 68.0 |

The equilibrium solubility of COMPOUND I (amorphous powder) in different media was determined by shaking for 72 h in an air bath shaker at 37° C.±0.5° C. as the test condition, as detailed in Table 4. The solubility of COMPOUND I is pH-dependent, almost insoluble at low pH (pH 1.0-5.0), very slightly soluble at pH 6.8, and sparingly soluble at pure water, requiring the use of reasonable solubilization means to increase its dissolution in vitro and in vivo.

TABLE 4
Equilibrium Solubility of COMPOUND I in Media
with Different pH Values

| Conditions | Equilibrium Solubility (ug/ml) |
|---|---|
| pH 1.0 | 0 |
| pH 2.0 | 0 |
| pH 3.0 | 0 |
| pH 3.5 | 0 |
| pH 4.0 | 0 |
| pH 4.5 | 0 |
| pH 5.0 | 0 |
| pH 6.0 | 1.5 |
| pH 6.8 | 217 |
| pH 7.0 | 365 |
| pH 8.0 | 20419 |
| H₂O (The final pH was around 9.3) | 22274 |

### Example 3. Selection of the Pulverization Process of the Drug Substance

Considering that the particle size of the API may affect the dissolution of the drug, the API was investigated for different pulverization processes (see Formulation 1 for mechanical pulverization and Formulation 4 for airflow pulverization). Considering the convenience and time efficiency of the process, the direct compression process of mixed powder was used for compression at the early stage of research and development. Two batches of tablets of 50 mg strength were prepared by the same formulation process, Prescribing Information are shown in Table 5.

TABLE 5
Formulation information of two batches of tablets
prepared by different API milling processes

| | Formulation 1 (Mechanical | | | | Formulation 4 (Drug Substance | | | |
|---|---|---|---|---|---|---|---|---|
| | milling of drug substance) | | | | Airflow Pulverization) | | | |
| | API Batch Number | | | | | | | |
| | NASH1-23-008S7-\textcircled{1} | | | | NASH1-23-008S7-WFH | | | |
| Particle Size | Mean | | | | Mean | | | |
| Distribution (μm) | Mv | D₁₀ | D₅₀ | D₉₀ | Mv | D₁₀ | D₅₀ | D₉₀ |
| | 22.6 | 2.2 | 12.6 | 52.6 | 20.0 | 1.4 | 9.0 | 49.6 |

| | Amount | Weight | Amount | Weight |
|---|---|---|---|---|
| Tablet components | (mg) | Ratio | (mg) | Ratio |
| API | 50 | 14.29% | 50 | 14.29% |
| Lactose (Granulac200) | 50 | 14.29% | 50 | 14.29% |
| Silicified | 80 | 22.86% | 80 | 22.86% |
| microcrystalline | | | | |
| celluloseSMCC50 | | | | |
| Silicified | 148 | 42.29% | 148 | 42.29% |
| microcrystalline | | | | |
| celluloseSMCC90 | | | | |
| CrospovidoneXL-10 | 15 | 4.29% | 15 | 4.29% |
| Colloidal silicon | 2 | 0.57% | 2 | 0.57% |
| dioxide | | | | |
| Sodium Stearyl | 5 | 1.43% | 5 | 1.43% |
| Fumarate | | | | |
| Total | 350 | 100.00% | 350 | 100.00% |

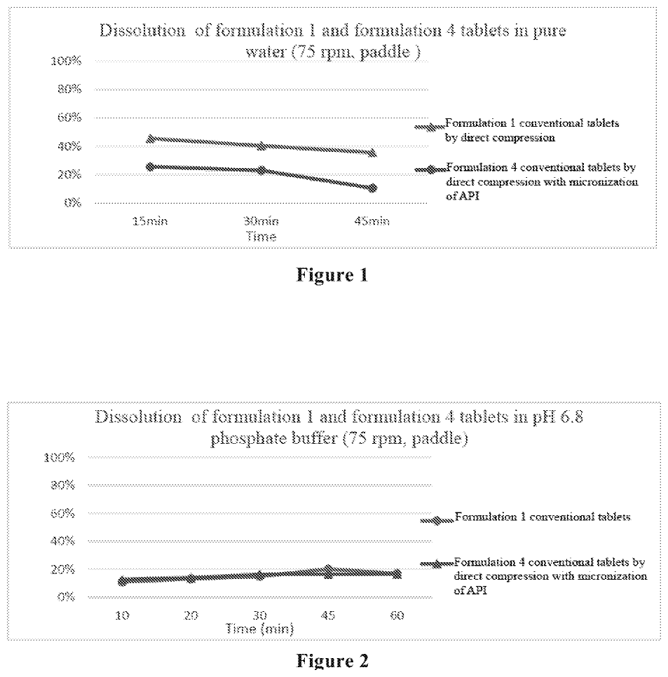

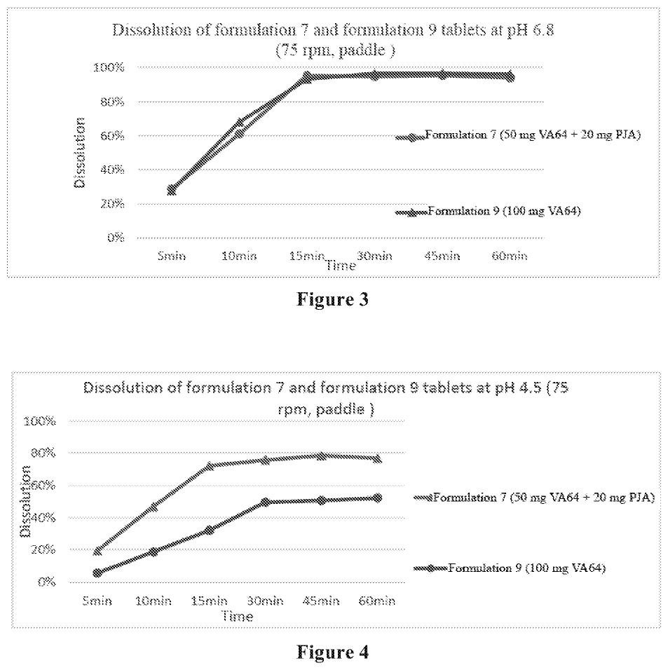

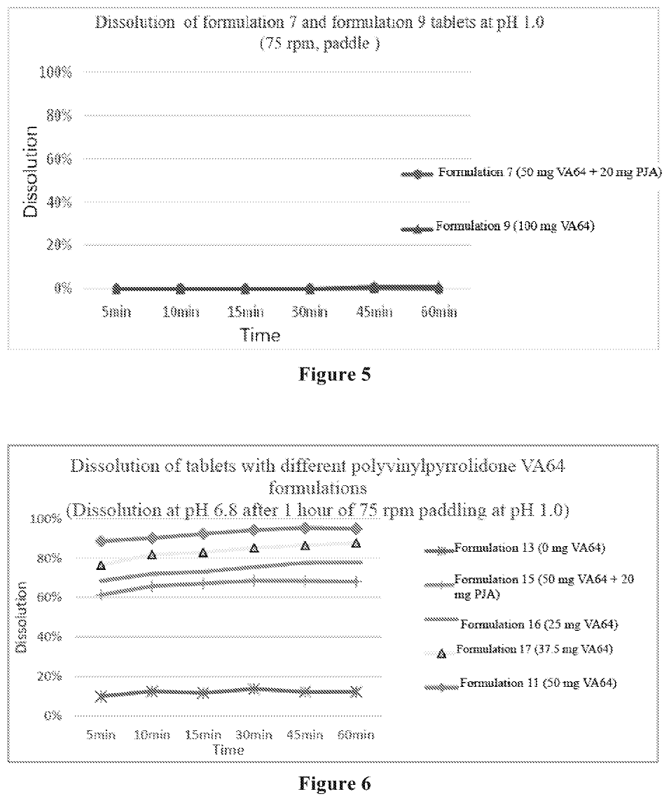

The dissolution profiles of these tablets were investigated in water and at pH 6.8 and are shown in FIGS. 1-2. It can be seen from the above results that the formulation 4 tablets made from the API by airflow pulverization did not have significant advantages in dissolution behavior over the formulation 1 tablets made from the API by mechanical pulverization, so the pulverization process of the API was determined as mechanical pulverization. The API particle size control reference range was tentatively limited to within 3 batch means+3σ (σ is the standard deviation), i.e., D50≤22.3 μm, D90≤68.0 μm.

### Example 4. Selection of Granulation Mode of COMPOUND I

The COMPOUND I tablets were prepared with dry granulation or direct powder compression process, which does not require solvents and can avoid the heating and drying step of wet granulation and other processes and reduce the risk of API degradation.

The flowability of the drug substance was studied, and the bulk and tapped densities of the drug substance are shown in Table 6.

TABLE 6
Loose density and tapped density of different batches of API

| API Lot No. | NASH1-23-008S7 | COMPOUND IC20001 |
|---|---|---|
| Usage | Early Formulation | Pilot registration batch study |
| | Discovery Studies | |
| API Handling | Untreated | Pass through 30 mesh sieve |
| | | after mechanical grinding |
| Bulk density | 0.48 g/ml | 0.33 g/ml |
| Solid density | 0.73 g/ml | 0.51 g/ml |
| Karl coefficient | 34.24 | 35.29 |
| Particle fluidity | Very poor flowability | Very poor flowability |

It can be seen from Table 6 that the API has very poor flowability whether it is milled or not. Direct powder compression requires good fluidity of raw materials and excipients in order to meet the requirements of uniformity of mixing and compression processes. After the dry granulation process is granulated through the screen mesh, the raw materials and excipients can be prepared into granules with better fluidity, reducing the risk of affecting product uniformity due to poor fluidity of the API.

### Example 5. Screening of the Amount of Solubilizing Excipient Polyvinylpyrrolidone VA64

It has been found early in the study that the solubility of COMPOUND I API is pH-dependent, almost insoluble at low pH conditions (pH 1.0-5.0), very slightly soluble at pH 6.8 and slightly soluble in pure water conditions. In contrast, the usual drug transport process in the human gastrointestinal tract is through the stomach with low pH (pH about 1.0-4.5) and then transported to the small intestine with high pH (pH about 4.5-6.8) for absorption. In order to reflect the disintegration and dissolution of COMPOUND I tablets in vivo more realistically, we chose more stringent dissolution conditions for the study. For the specific operation, we refer to the Chinese Pharmacopoeia 2015 Edition IV 0931 Dissolution and Release Determination of Enteric Formulations Method I. After stirring for 1 hour at 75 rpm in 750 ml of pH 1.0 hydrochloride buffer, 250 ml of sodium phosphate solution was added to adjust the medium to pH 6.8 for dissolution testing (hereafter referred to as the dissolution method of pH 1.0 followed by pH 6.8).

The screening of polyvinylpyrrolidone VA64 dosage and the investigation of glucosamine dosage in COMPOUND I tablet prescription was carried out using the 50 mg size tablet prepared by mixing the granules with the added excipients after dry granulation, and the designed prescription is shown in Table 7.

TABLE 7
Prescription 10-17 tablet dosage information

| | Formulation Information | | | | | | | | | |
|---|---|---|---|---|---|---|---|---|---|---|
| | Formulation 10 | | Formulation 11 | | Formulation 12 | | Formulation 13 | | Formulation 14 | |
| | Amt | | Amt | | Amt | | Amt | | Amt | |
| Name | (mg) | Wt % | (mg) | Wt % | (mg) | Wt % | (mg) | Wt % | (mg) | Wt % |
| API (intragranular) | 50 | 12.99% | 50 | 12.99% | 50 | 12.99% | 50 | 24.51% | 50 | 24.51% |
| VA64 | 25 | 6.49% | 50 | 12.99% | 25 | 6.49% | / | / | 10 | 4.90% |
| (intragranular) | | | | | | | | | | |
| Glucosamine | 10 | 2.60% | / | / | 20 | 5.19% | / | / | / | / |
| (intragranular) | | | | | | | | | | |
| Mannitol | 85 | 22.08% | 70 | 18.18% | 75 | 19.48% | 74 | 36.27% | 74 | 36.27% |
| (intragranular) | | | | | | | | | | |
| PROSOLV | 100 | 25.97% | 100 | 25.97% | 100 | 25.97% | 60 | 29.41% | 50 | 24.51% |
| SMCC90 | | | | | | | | | | |
| (intragranular) | | | | | | | | | | |
| XL-10 | 10 | 2.60% | 10 | 2.60% | 10 | 2.60% | 8 | 3.92% | 8 | 3.92% |
| (intragranular) | | | | | | | | | | |
| Colloidal silica | 1 | 0.26% | 1 | 0.26% | 1 | 0.26% | / | / | / | / |
| (intragranular) | | | | | | | | | | |
| Sodium stearyl | 3.5 | 0.91% | 3.5 | 0.91% | 3.5 | 0.91% | 4 | 1.96% | 4 | 1.96% |
| fumarate | | | | | | | | | | |
| (intragranular) | | | | | | | | | | |
| Silicified | 93 | 24.16% | 93 | 24.16% | 93 | 24.16% | / | / | / | / |
| microcrystalline | | | | | | | | | | |
| cellulosePROSOLV | | | | | | | | | | |
| SMCC90HD | | | | | | | | | | |
| (extragranular) | | | | | | | | | | |
| Cross-linked | 5 | 1.30% | 5 | 1.30% | 5 | 1.30% | 4 | 1.96% | 4 | 1.96% |
| povidone XL-10 | | | | | | | | | | |
| (extragranular) | | | | | | | | | | |
| Colloidal silica | 1 | 0.26% | 1 | 0.26% | 1 | 0.26% | / | / | / | / |
| (extragranular) | | | | | | | | | | |
| Sodium stearyl | 1.5 | 0.39% | 1.5 | 0.39% | 1.5 | 0.39% | 4 | 1.96% | 4 | 1.96% |
| fumarate | | | | | | | | | | |
| (extragranular) | | | | | | | | | | |
| Total | 385 | 100.00% | 385 | 100.00% | 385 | 100.00% | 204 | 100.00% | 204 | 100.00% |

| | Formulation Information | | | | | |
|---|---|---|---|---|---|---|
| | Formulation 15 | | Formulation 16 | | Formulation 17 | |
| | Amt | | Amt | | Amt | |
| Name | (mg) | Wt % | (mg) | Wt % | (mg) | Wt % |
| API (intragranular) | 50 | 19.69% | 50 | 17.90% | 50 | 16.34% |
| VA64 | 10 | 3.94% | 25 | 8.95% | 37.5 | 12.25% |
| (intragranular) | | | | | | |
| Glucosamine | 20 | 7.87% | / | / | / | / |
| (intragranular) | | | | | | |
| Mannitol | 64 | 25.20% | 75 | 26.85% | 86.5 | 28.27% |
| (intragranular) | | | | | | |
| PROSOLV | 90 | 35.43% | 97 | 34.72% | 97 | 31.70% |
| SMCC90 | | | | | | |
| (intragranular) | | | | | | |
| XL-10 | 8 | 3.15% | 15 | 5.37% | 15 | 4.90% |
| (intragranular) | | | | | | |
| Colloidal silica | | / | 1.375 | 0.49% | 1 | 0.33% |
| (intragranular) | | | | | | |
| Sodium stearyl | 4 | 1.57% | 6 | 2.15% | 9 | 2.94% |
| fumarate | | | | | | |
| (intragranular) | | | | | | |
| Silicified | / | / | / | / | / | / |
| microcrystalline | | | | | | |
| cellulosePROSOLV | | | | | | |
| SMCC90HD | | | | | | |
| (extragranular) | | | | | | |
| Cross-linked | 4 | 1.57% | 7.5 | 2.68% | 7.5 | 2.45% |
| povidone XL-10 | | | | | | |
| (extragranular) | | | | | | |
| Colloidal silica | / | / | 1 | 0.36% | 1 | 0.33% |
| (extragranular) | | | | | | |
| Sodium stearyl | 1 | 1.57% | 1.5 | 0.54% | 1.5 | 0.49% |
| fumarate | | | | | | |
| (extragranular) | | | | | | |
| Total | 254 | 100.00% | 279.375 | 100.00% | 306 | 100.00% |

After stirring for 1 h in pH 1.0 hydrochloric acid solution using the 75 rpm paddle method, and found that the dissolution was <1% for all batches, which was consistent with the equilibrium solubility results in pH 1.0 hydrochloric acid solution. The dissolution solution was adjusted to pH 6.8 by continued addition of phosphate and dissolution curve studies were conducted to examine the amount of polyvinylpyrrolidone VA64 needed by Formulations 11, 13, 15, 16 and 17. The results are shown in FIG. 6.

The results showed that the higher the dosage of polyvinylpyrrolidone VA64 in 50 mg size COMPOUND I tablets, the better the dissolution effect. 50 mg of polyvinylpyrrolidone VA64 can make the dissolution of 50 mg tablets greater than 90%.

### Example 6. The Effect of Glucosamine

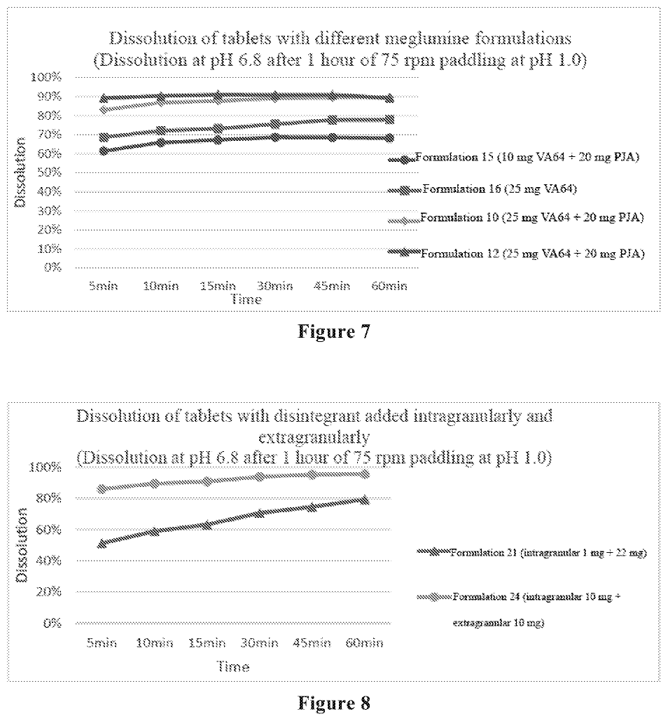

The examination of glucosamine dosage in COMPOUND I tablet formulations was carried out, and the results are shown in Table 7, and the glucosamine dosage was examined by Formulations 10, 12, 15 and 16. The dissolution behavior of the above tablets under dissolution conditions (dissolution method of pH 1.0 then pH 6.8) is shown in FIG. 7.

The results showed that the addition of glucosamine at 25 mg of polyvinylpyrrolidone VA64 improved the dissolution of COMPOUND I tablets, but the solubilization-enhancing effect was less pronounced than that of polyvinylpyrrolidone VA64 in Formulations 16, 10 and 12.

During the pilot study, one-month influence factor studies were conducted under sealing and opening conditions based on tablets of Formulations 11, 13 and 15 packaged using HDPE bottles. The influence factor studies were examined under strong light (25° C., 4500 Lx±500 Lx), high temperature and high humidity (40° C., RH 75%), high humidity (25° C., RH 92.5%), and high temperature 60° C. conditions. The results are shown in Table 8.

TABLE 8
One-month influencing factors study of tablets of small trial formulation 11, 13 and 15

| | | Condition | | | | | | |
|---|---|---|---|---|---|---|---|---|
| | | | | | HighTemperature | | | |
| | | | | HighTemperature | and humidity | | | |
| | | | HighTemperature | 25° C., | 40° C., | 40° C., | | |
| | | | 60° C. | RH92.5% | RH75% | RH75% | Illumination | |
| Test Item | | Day 0 | Packaged | Unpackaged | Unpackaged | Packaged | Unpackaged | Packaged |
| Related | Purity | 99.741 | 99.475 | 99.741 | 99.650 | 99.765 | 99.793 | 99.711 |
| Substance % | COMPOUND | 0.010 | 0.067 | 0.012 | 0.055 | 0.010 | 0.013 | ND |
| (Formulation 11) | I-B | | | | | | | |
| | COMPOUND | 0.087 | 0.087 | 0.093 | 0.097 | 0.090 | 0.087 | 0.093 |
| | I-IM1 | | | | | | | |
| | COMPOUND | ND | ND | ND | ND | ND | ND | ND |
| | I-C | | | | | | | |
| | COMPOUND | ND | ND | ND | ND | ND | ND | ND |
| | I-IM2 | | | | | | | |
| | Maximum | 0.061 | 0.081 | 0.062 | 0.048 | 0.051 | 0.041 | 0.064 |
| | unknown | | | | | | | |
| | single impurity | | | | | | | |
| | Total Impurity | 0.259 | 0.525 | 0.259 | 0.350 | 0.235 | 0.207 | 0.289 |
| Related | Purity | 99.505 | 99.695 | 99.752 | 99.571 | 99.452 | 99.805 | 99.584 |
| Substance % | COMPOUND | ND | 0.009 | 0.013 | 0.107 | 0.009 | 0.010 | 0.009 |
| (Formulation 13) | I-B | | | | | | | |
| | COMPOUND | 0.093 | 0.096 | 0.089 | 0.092 | 0.090 | 0.091 | 0.096 |
| | I-IM1 | | | | | | | |
| | COMPOUND | ND | ND | ND | ND | ND | ND | ND |
| | I-C | | | | | | | |
| | COMPOUND | ND | ND | ND | ND | ND | ND | ND |
| | I-IM2 | | | | | | | |
| | Maximum | 0.069 | 0.091 | 0.050 | 0.103 | 0.094 | 0.044 | 0.056 |
| | unknown | | | | | | | |
| | single impurity | | | | | | | |
| | Total Impurity | 0.495 | 0.305 | 0.248 | 0.429 | 0.548 | 0.195 | 0.416 |
| Related | Purity | 99.752 | 99.726 | 99.752 | 99.686 | 99.680 | 99.630 | 99.597 |
| Substance % | COMPOUND | 0.014 | 0.012 | 0.009 | ND | ND | 0.018 | ND |
| (Formulation 15) | I-B | | | | | | | |
| | COMPOUND | 0.091 | 0.095 | 0.093 | 0.093 | 0.088 | 0.092 | 0.092 |
| | I-IM1 | | | | | | | |
| | COMPOUND | ND | ND | ND | ND | ND | ND | ND |
| | I-C | | | | | | | |
| | COMPOUND | ND | ND | ND | ND | ND | ND | ND |
| | I-IM2 | | | | | | | |
| | Maximum | 0.034 | 0.053 | 0.028 | 0.034 | 0.066 | 0.128 | 0.131 |
| | unknown | | | | | | | |
| | single impurity | | | | | | | |
| | Total Impurity | 0.248 | 0.274 | 0.248 | 0.314 | 0.320 | 0.370 | 0.403 |

The results indicate that (1) the test tablets were generally stable in the one-month stability test.

2. Formulation 15 tablets containing glucosamine produced significant impurities (>0.1%) under both unpackaged and packaged conditions under illumination at 4500 Lx±500 Lx, so glucosamine was not used in the subsequent study.

3. Unpackaged Formulation 13 tablets produced impurities (>0.1%) under high temperature and high humidity (40° C., RH75%) conditions, which were slightly but not significantly reduced after packaging.

4. Formulation 11 tablets with packaging containing 50 mg of polyvinylpyrrolidone VA64 showed an increase in total impurities after one month at 60° C., but the maximum unknown single impurity did not change significantly and was still less than 0.1% (0.081%), indicating that after one month at 60° C., the tablets can degrade more small impurities, so the storage conditions of the samples need to avoid intense high temperature environment.

Unpackaged Formulation 13 tablets without polyvinylpyrrolidone VA64, impurity B and the largest unknown single impurity became slightly larger after one month at high temperature and high humidity (40° C., RH75%). While the unpackaged and packed Formulation 11 tablets containing 50 mg of polyvinylpyrrolidone VA64 did not change significantly at high temperature and high humidity (40° C., RH75%), which shows that polyvinylpyrrolidone VA64 can improve the stability of COMPOUND I API.

In conclusion, although glucosamine can slightly improve the dissolution ability of COMPOUND I tablets, Formulation 15 tablets containing glucosamine produced significant impurities under packaged and unpackaged conditions during a one-month illumination stability (4500Lx±500Lx) study. The study showed that polyvinylpyrrolidone VA64 improves the stability of COMPOUND I.

### Example 7. Selection of Disintegrant Type and Dosage

The compatibility test of API and excipients and the one-month stability stress test of Formulations 11, 13, and 15 lab-scale tablets confirmed that the combination of crospovidone XL-10 and COMPOUND I drug substance had good stability.

Table 9 shows the effect of intragranular and extragranular dosages of crospovidone XL-10. The information of Formulation 21 and Formulation 24 tablets are shown in Table 10. The study found that due to the viscosity of COMPOUND I during dissolution, the tablets could not quickly disintegrate even with a sufficient dosage of disintegrant. Very slow dissolved during dissolution in pH 4.5 acetate buffer and pH 6.8 phosphate buffer. In pH 1.0 hydrochloric acid, the tablets were still intact but cracked by gently touch after 1 h of dissolution.

TABLE 9
Investigation of internal and external dosage of crospovidone XL-10

| | | | | Dissolution |
|---|---|---|---|---|
| | | | Dissolution | status of |
| 47 | | Intragranular and | status of | tablets at |
| Disintegrant | Formulation | extragranular dosages | tablets at pH 1.0 | pH 4.5/6.8 |
| Crospovidone | Formulation 10 | Intragranular 10 mg + extragranular 5 mg | Tablets were still | Very slow |
| XL-10 | Formulation 11 | | intact but cracked by | dissolved |
| | Formulation 12 | | gently touch after 1 h | during |
| | Formulation 13 | Intragranular 8 mg + extragranular 4 mg | of dissolution. | dissolution |
| | Formulation 15 | Intragranular 8 mg + extragranular 5 mg | | |
| | Formulation 16 | Intragranular 15 mg + extragranular 7.5 mg | | |
| | Formulation 17 | | | |
| | Formulation 21 | Intragranular 1 mg + extragranular 22 mg | | |
| | Formulation 24 | Intragranular 10 mg + extragranular 10 mg | | |

TABLE 10
Composition of Formulation 21 and Formulation 24 Tablets

| | Formulation 21 | | Formulation 24 | |
|---|---|---|---|---|
| Name of drug substance and excipients | Dosage (mg) | Ratio | Dosage (mg) | Ratio |
| Drug substance (intragranular) | 50 | 21.46% | 50 | 25.00% |
| Polyvinylpyrrolidone VA64 (intragranular) | 50 | 21.46% | 50 | 25.00% |
| Mannitol (intragranular) | 50 | 21.46% | 40 | 20.00% |
| PROSOLV SMCC90 (intragranular) | 50 | 21.46% | 30 | 15.00% |
| Crospovidone XL-10 (intragranular) | 1 | 0.43% | 10 | 5.00% |
| Sodium stearyl fumarate (intragranular) | 9 | 3.86% | 9 | 4.50% |
| Crospovidone XL-10 (extragranular) | 22 | 9.44% | 10 | 5.00% |
| Sodium stearyl fumarate (extragranular) | 1 | 0.43% | 1 | 0.50% |
| Total | 233 | 100.00% | 200 | 100.00% |

The dissolution behavior of Formulation 21 and Formulation 24 tablets was investigated under more stringent dissolution conditions (dissolution method at pH 1.0 followed by pH 6.8), and the results are shown in FIG. 8.

The results showed that (1) the amount of disintegrant reached a higher level, but still did not achieve the effect of faster disintegration, which may be related to the properties of COMPOUND I; (2) higher dissolution rate is achieved by adding disintegrant both before and after granulation; and (3) tablets with cross-linked polyvidone XL-10 was more stable in the one-month pilot stability study.

### Example 8. Selection of Lubricant Dosage

In the early study, the target tablet weight was designed as 385 mg (such as in Formulation 10, Formulation 11, and Formulation 12). Due to the large dosage of filler, even if the dosage of sodium stearyl fumarate was controlled at a low level, the dry granulation process was smooth, and there was no sticking roller phenomenon. Reducing the target tablet weight to about 200 mg and increasing the dosage of sodium stearyl fumarate could effectively solve the skicking roller phenomenon of dry granulation, as shown in Table 11.

TABLE 11
Selection of sodium stearyl fumarate dosage

| | Dosage of Sodium | Tablet | |
|---|---|---|---|
| Formulation | Stearyl Fumarate | Weight | Process |
| 10 | Intragranular 3.5 mg + | 385 mg | Dry granulation without sticking rollers |
| | extragranular 1.5 mg | | |
| 11 | Intragranular 3.5 mg + | 385 mg | Dry granulation without sticking rollers |
| | extragranular 1.5 mg | | |
| 12 | Intragranular 3.5 mg + | 385 mg | Dry granulation without sticking rollers compacted |
| | extragranular 1.5 mg | | |
| 13 | Intragranular 4 mg + | 204 mg | Dry granulation with sticking rollers (severe) |
| | extragranular 4 mg | | |
| 15 | Intragranular 4 mg + | 254 mg | Dry granulation with sticking rollers (severe) |
| | extragranular 4 mg | | |
| 16 | Intragranular 6 mg + | 280 mg | Dry granulation with sticking rollers (mild) |
| | extragranular 1.5 mg | | |
| 17 | Intragranular 9 mg + | 306 mg | Dry granulation without sticking rollers |
| | extragranular 1.5 mg | | (in good conditions) |
| 21 | Intragranular 9 mg + | 233 mg | Dry granulation without sticking rollers |
| | extragranular 1 mg | | (in good conditions) |
| 24 | Intragranular 9 mg + | 200 mg | Dry granulation without sticking rollers |
| | extragranular 1 mg | | (in good conditions) |

In Formulation 21 and Formulation 24 (Table 10), which had a higher internal amount of sodium stearyl fumarate, the colloidal silica was removed from the formulation, and the dry granulation process was still smooth, so colloidal silica was not added in the subsequent study.

### Example 9. Screening of Mannitol Type and Dosage

During the early studies, two types of mannitol, 200SD from Roget, France, and M100 from Merck, Germany, were examined, both of which had better formulation characteristics and could meet the development of 50 mg size COMPOUND I tablets. Considering the high requirement of tablet content uniformity for 5 mg small size dose tablets, mannitol M100 (with an average particle size of 100 microns), which has a smaller average particle size and is closer to other excipients, was used as the final mannitol model so that the mixing process could be more uniform. The content uniformity of all three batches registered in the pilot test for 50 mg and 5 mg size tablets met the requirements. Formulation accounted for a relatively large proportion of excipients and raw materials particle size information is shown in Table 12.

TABLE 12
The raw material and particle size information for a larger proportion of
excipients and API size

| Name | Manufacturer | Size | Function |
|---|---|---|---|
| API (Intragranular) | Gannex | D90 ≈ 42.0 μ M | API |
| Polyvinylpyrrolidone VA64 (Intragranular) | BASF | D90 ≈ 100 μ M | Solubilizing |
| | | | effector |
| Mannitol (200SD) (Intragranular) | Roget, France | Average particle size: approximate | Filler |
| | | 200 μ m | |
| Mannitol (M100) (Intragranular) | Merck, Germany | Average particle size: approximate | Filler |
| | | 100 μ m | |
| PROSOLV SMCC90 (Intragranular) | JRS | Average particle size: 125 μ m | Filler |

### Example 10. Determination of Tablet Formulation Processes

On the basis of development of the formulation process, the 50 mg and 5 mg COMPOUND I tablets were developed and determined using the dissolution profiles and stability as parameters. The optimized lab-scale formulations of 50 mg and 5 mg are shown in Table 13-1 and Table 13-2. The process control is shown in Table 14-1 and Table 14-2. The difference between 5 mg tablets and 50 mg tablets was only the difference of API dosage. The types and dosage of other excipients were consistent with those of 50 mg tablets, and the process of both were consistent.

TABLE 13-1
Optimized lab-scale formulation of 50 mg COMPOUND I tablets
(Formulation 24)

| | Theoretical | | |
|---|---|---|---|
| | Dosage (g) | | |
| Ingredient | (1000 Tablets) | Overage | Function |
| COMPOUND I | 50 | N/A | Active |
| | | | pharmaceutical |
| | | | ingredient |
| Silicified | 30 | N/A | Filler |
| microcrystalline | | | |
| cellulose SMCC90 | | | |
| Polyvinylpyrrolidone | 50 | N/A | Solubilizer |
| VA64 | | | |
| Mannitol M100 | 40 | N/A | Filler |
| Crospovidone XL-10 | 10 | N/A | Disintegrant |
| Sodium stearyl | 9 | N/A | Lubricant |
| fumarate | | | |
| Crospovidone XL-10 | 10 | N/A | Disintegrant |
| Sodium stearyl fumarate | 1 | N/A | Lubricant |
| Total weight | 200 | | |

TABLE 13-2
Optimized lab-scale formulation of 5 mg COMPOUND I tablets
(Formulation 26)

| | Theoretical | | |
|---|---|---|---|
| | Dosage (g) | | |
| | (1000 | | |
| Ingredient | Tablets) | Overage | Function |
| COMPOUND I | 5 | N/A | Active pharmaceutical |
| | | | ingredient |
| Silicified microcrystalline | 30 | N/A | Filler |
| cellulose SMCC90 | | | |
| Polyvinylpyrrolidone VA64 | 50 | N/A | Solubilizer |
| Mannitol M100 | 40 | N/A | Filler |
| Crospovidone XL-10 | 10 | N/A | Disintegrant |
| Sodium stearyl fumarate | 9 | N/A | Lubricant |
| Crospovidone XL-10 | 10 | N/A | Disintegrant |
| Sodium stearyl fumarate | 1 | N/A | Lubricant |
| Total weight | 155 | | |

TABLE 14-1
process of 50 mg COMPOUND I tablets (Formulation 24)

| Process Step | Process Description |
|---|---|
| Pretreatment of drug | Silicified microcrystalline cellulose SMCC90, Polyvinylpyrrolidone VA64, mannitol M100, |
| substance and | crospovidone XL-10, and sodium stearyl fumarate were all sifted through a 40-mesh sieve and |
| excipients | COMPOUND I was sifted through a 30-mesh sieve to remove the possible agglomerates of |
| | materials generated during storage. |
| Pre-blending | The materials were blended manually in a ziplock bag for 10 min. |
| Dry granulation | A TF-MINI_ITN dry granulator was used for granulation. The roller speed and feed screw speed |
| | were adjusted. |
| Final blending | The materials were blended manually in a ziplock bag for 10 min. |
| Tableting | A high-speed tablet press machine was used for tableting. The target weight of COMPOUND I |
| | tablet was controlled at 200 mg. The weight difference of single COMPOUND I tablets was |
| | controlled within ± 7.5%, the average tablet weight difference was controlled within ± 5.0%. |

TABLE 14-2
process of 5 mg COMPOUND I tablets (Formulation 26)

| Process Step | Process Description |
|---|---|
| Pretreatment of drug | Silicified microcrystalline cellulose SMCC90, Polyvinylpyrrolidone VA64, mannitol M100, |
| substance and | crospovidone XL-10, and sodium stearyl fumarate were all sifted through a 40-mesh sieve and |
| excipients | COMPOUND I was sifted through a 30-mesh sieve to remove the possible agglomerates of |
| | materials generated during storage. |
| Pre-blending | The materials were blended manually in a ziplock bag for 10 min. |
| Dry granulation | A TF-MINI_ITN dry granulator was used for granulation. The roller speed and feed screw speed |
| | were adjusted. |
| Final blending | The materials were blended manually in a ziplock bag for 10 min. |
| Tableting | A high-speed tablet press machine was used for tableting. The target weight of COMPOUND I |
| | tablet was controlled at 155 mg. The weight difference of single COMPOUND I tablets was |
| | controlled within ± 7.5%, the average tablet weight difference was controlled within ± 5.0%. |

### 1. Dissolution Study of 50 mg Tablets by Verified Formulation Processes

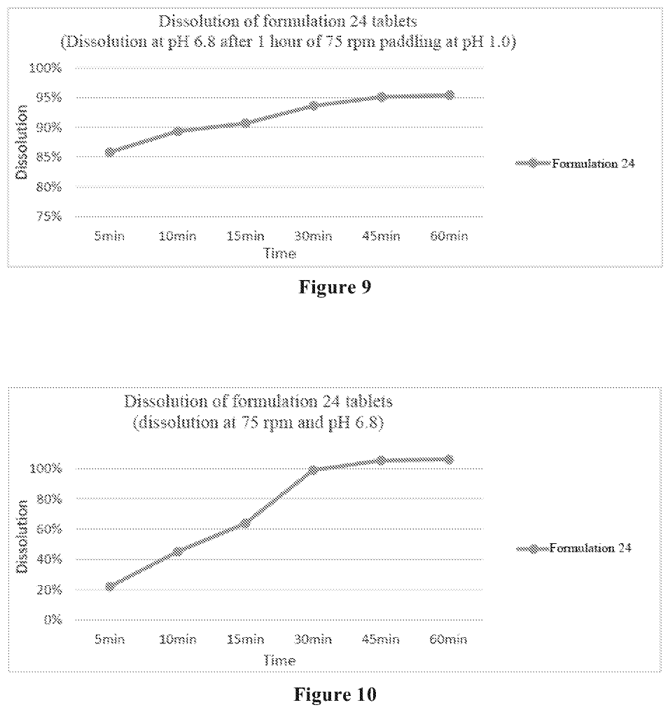

Dissolution profile of 50 mg tablets prepared by optimized formulation (Formulation 24) process was studied in dissolution medium under more stringent dissolution conditions (dissolution method at pH 1.0 followed by pH 6.8) and dissolution method at pH 6.8. The results are shown in FIGS. 9-10.

### 2. Dissolution Study of 5 mg Tablets by Verified Formulation Processes

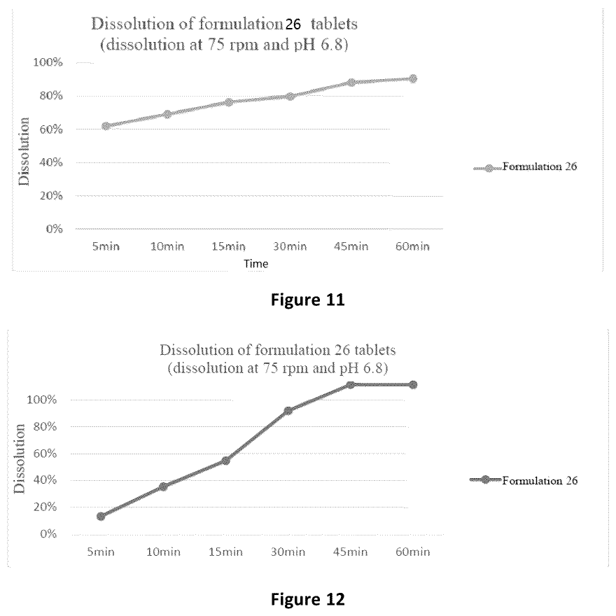

Dissolution curve of 5 mg tablets prepared by optimized formulation (Formulation 26) was studied in dissolution medium under stricter dissolution conditions (dissolution method at pH 1.0 followed by pH 6.8) and dissolution method at pH 6.8. The results are shown in FIGS. 11-12.

In summary, the results of lab-scale development of COMPOUND I tablets showed that 50 mg and 5 mg strength of COMPOUND I tablets could effectively increase the dissolution of COMPOUND I using 50 mg of polyvinylpyrrolidone VA64 as a solubilizing agent. 30 mg silicified microcrystalline cellulose SMCC90 and 40 mg mannitol were used as fillers, 20 mg cross-linked polyvinyl XL-10 was used as disintegrant, and 10 mg sodium stearyl fumarate was used as lubricant. During the dry granulation and compression process, the process is stable and controllable and produces tablets of acceptable quality, as expected for further development.

### 3. In Vivo Study of 50 mg Tablets by Verified Formulation Processes (Formulation 24) in Beagle Dogs

Formulation 24 and formulation 27 tablets by the same dry granulation and tableting processes were chosen for the in vivo comparative study in Beagle dogs. The formulations are shown in Table 15.

TABLE 15
Formulation Information for Formulation 24 and Formulation 27
Tablets

| Formulation | Formulation 24 | | Formulation 27 | |
|---|---|---|---|---|
| Name of drug substance and excipients | Amount (mg) | Weight % | Amount (mg) | Weight % |
| Drug substance (intragranular) | 50 | 25.00% | 50 | 33.33% |
| Copovidone VA64 (intragranular) | 50 | 25.00% | 0 | 0.00% |
| Mannitol (intragranular) | 40 | 20.00% | 40 | 26.67% |
| PROSOLV SMCC90 (intragranular) | 30 | 15.00% | 30 | 20.00% |
| Crospovidone XL-10 (intragranular) | 10 | 5.00% | 10 | 6.67% |
| Sodium stearyl fumarate (intragranular) | 9 | 4.50% | 9 | 6.00% |
| Crospovidone XL-10 (extragranular) | 10 | 5.00% | 10 | 6.67% |
| Sodium stearyl fumarate (extragranular) | 1 | 0.50% | 1 | 0.67% |
| Total | 200 | 100.00% | 150 | 100% |

Results of the in vivo comparative study in beagle dogs are shown in Table 16.

TABLE 16
Results of the in vivo Beagle Dog Comparison Study of Formulation
24 and Formulation 27 Tablets

| Sample | Formulation 24 | Formulation 27 |
|---|---|---|
| Dosage | 50 mg/Beagle dog | 50 mg/Beagle dog |
| Maximum Blood | 7,763 ± 1,760 | 2,937 ± 1,984 |
| Concentration Cₘₐₓ (ng/ml) | | |
| In-Vivo Exposure | 33,835 ± 11,589 | 18,985 ± 10,531 |
| AUC₀₋ₜ (ng.h/mL) | | |

The above results showed that the maximum blood concentration, exposure and bioavailability of Formulation 24 tablets was significantly higher than that of Formulation 27 tablets, Formulation 24 tablets have a better homogeneity, presenting obvious advantages.

4. Selection of Dissolution Conditions with Distinctiveness for 50 mg COMPOUND I Tablets

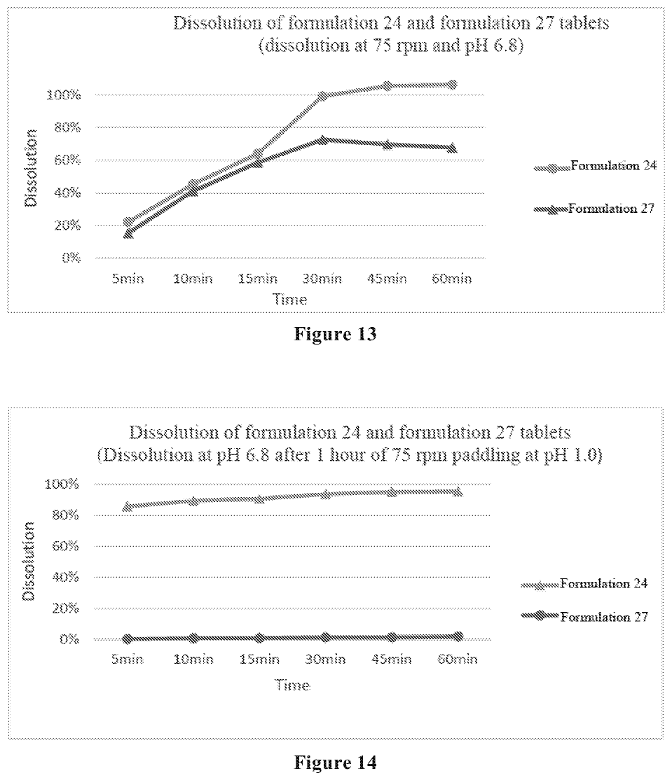

Dissolution condition of phosphate buffer (pH 6.8) and a more stringent dissolution condition (dissolution method of pH 1.0 first and then pH 6.8) were chosen to study the dissolution of Formulation 24 and Formulation 27. Results are shown in FIG. 13 and FIG. 14.

The above results indicated that the dissolution profiles of Formulation 24 and Formulation 27 tablets in phosphate buffer (pH 6.8) reflected the difference of these two formulations in Beagle dogs more closely. The two dissolution profile results also matched the in vivo exposure and bioavailability in Table 11 and were clearly distinguished at 45 min. Under more stringent dissolution conditions (dissolution method of pH 1.0 first and then pH 6.8), Formulation 27 COMPOUND I tablets failed to dissolve and could not reflect the dissolution and absorption of COMPOUND I tablets in Beagle dogs, indicating that the condition was excessively stringent.

In summary, the dissolution conditions were determined as phosphate buffer (pH 6.8) at a speed of 75 rpm for 45 min, where the evaluation criterion of dissolution of greater than 75% could be achieved.

### Example 11. Properties of the Tablet Formulation

During the development of the COMPOUND I tablet formulation, the amount of impurities, dissolution profile, and crystal form were studied.

### The Impurities

Table 17 shows the amount of impurities in the API and in three pilot batches of 50 mg tablets. Table 18 shows the amount of impurities in the API and in three pilot batches of 5 mg tablets. The amount of impurities in each batch of samples were relatively stable and no significant change in the amount and type of impurities was observed after the tableting process.

TABLE 17
Comparison of Related Substances Results between Three Pilot Batches of COMPOUND I
Tablets (50 mg) and API

| | COMPOUND I | | | |
|---|---|---|---|---|
| | Drug | COMPOUND I | COMPOUND I | COMPOUND I |
| | Substance Batch | Tablets | Tablets | Tablets |
| | COMPOUND | Batch | Batch | Batch |
| Criteria for Tablets | IC20001 | R4220001 | R4220002 | R4220003 |
| COMPOUND I-B should be NMT 0.20% | 0.015% | 0.013% | 0.013% | 0.013% |
| COMPOUND I-IM1 should be NMT 0.15% | 0.12% | 0.12% | 0.12% | 0.12% |
| COMPOUND I-C should be NMT 0.15% | Not detected | Not detected | Not detected | Not detected |
| COMPOUND I-IM2 should be NMT 0.15% | 0.12% | 0.12% | 0.12% | 0.12% |
| Any individual unspecified impurity | 0.048% | 0.04% | 0.04% | 0.03% |
| should be NMT 0.2% | | | | |
| Total impurities should be NMT 2.0% | 0.47% | 0.46% | 0.47% | 0.47% |

TABLE 18
Comparison of Related Substances Results between Three Pilot Batches of COMPOUND I Tablets (5 mg) and API

| | COMPOUND I | | | |
|---|---|---|---|---|
| | Drug Substance | COMPOUND I | COMPOUND I | COMPOUND I |
| | Batch | Tablets | Tablets | Tablets |
| | COMPOUND | R4220004 | R4220005 | R4220006 |
| Criteria for Tablets | IC20001 | batch | batch | batch |
| COMPOUND I-B should be NMT 0.20% | 0.015% | 0.014% | 0.015% | 0.014% |
| COMPOUND I-IM1 should be NMT | 0.12% | 0.12% | 0.12% | 0.12% |
| 0.15% | | | | |
| COMPOUND I-C should be NMT 0.15% | Not detected | Not detected | Not detected | Not detected |
| COMPOUND I-IM2 should be NMT | 0.12% | 0.12% | 0.12% | 0.12% |
| 0.15% | | | | |
| Any individual unspecified impurity should | 0.048% | 0.04% | 0.04% | 0.04% |
| be NMT 0.2% | | | | |
| Total impurities should be NMT 2.0% | 0.47% | 0.48% | 0.47% | 0.47% |

Note:
Percentage of related substance = (amount of related substance/amount of COMPOUND I) × 100%. Both the amounts of COMPOUND I in the API and tablets were determined using the area normalization method and the same measurement methods (mobile phase system and ratio, chromatographic column, injection volume, etc.)

### 2. Comparison of Dissolution Profile

Dissolution profiles of three batches of 50 mg and 5 mg tablets were studied (batch No. of 50 mg tablets: R4220001, R4220002, and R4220003; batch No. of 5 mg tablets: R4220004, R4220005, and R4220006).

### 2.1 Dissolution Conditions

Dissolution medium: pH 6.8 phosphate buffer
Method: Paddle Method.
Rotate speed: 75 rpm.

Sampling points: Samples were taken at the following time points, 5, 10, 15, 30, 45 and 60 min. The subsequent filtrate was taken as the sample solution, and the same amount of the dissolution medium at the same temperature was added.

Sampling points: Samples were taken at the following time points, 5, 10, 15, 30, 45 and 60 min. The subsequent filtrate was taken as the sample solution, and the same amount of the dissolution medium at the same temperature was added.

| | | Rotate | |
|---|---|---|---|
| | | Speed | Sampling Time |
| No. | Dissolution Medium | (rpm) | Point (min) |
| 1 | Acetate buffer containing | 75 | 5, 10, 15, 30, 45, 60, |
| | 1.0% SLS (pH 4.5) | | 90, 120 |
| 2 | Phosphate buffer (pH 6.0) | 75 | 5, 10, 15, 30, 45, 60 |
| 3 | Phosphate buffer (pH 6.8) | 75 | 5, 10, 15, 30, 45, 60 |
| 4 | pure water | 75 | 5, 10, 15, 30, 45, 60 |

Note: Sodium lauryl sulfate is hereinafter referred to as SLS.

### 2.2 Method

HPLC column (recommended column: Halo C18, 100×4.6 mm, 2.7 m) was used. Mobile phase A was 0.001% trifluoroacetic acid in water. Mobile phase B was 0.001% trifluoroacetic acid in acetonitrile. Flow rate 1.0 ml/min, column temperature of 40 degrees C., detection wavelength 230 nm. Elution is done as shown in the table below.

| time (min) | Mobile phase A (%) | Mobile Phase B (%) |
|---|---|---|
| 0 | 70 | 30 |
| 4 | 30 | 70 |
| 9 | 10 | 90 |
| 9.1 | 0 | 100 |
| 12 | 0 | 100 |
| 12.1 | 70 | 30 |
| 15 | 70 | 30 |

### 2.3 Results

The dissolution rates of three batches of 50 mg COMPOUND I tablets (batch numbers: R4220001, R4220002, R4220003) were shown in Tables 15-18. The dissolution rates of 3 batches of samples of COMPOUND I tablets with 5 mg strength (batch numbers: R4220004, R4220005 and R4220006) were shown in Tables 19-22.

TABLE 19
Summary of dissolution results of COMPOUND I tablets
(50 mg) in phosphate solution (pH 6.8) (n = 12)

| | COMPOUND I Tablets (Q ± SD, %) | | |
|---|---|---|---|
| Time | R4220001 | R4220002 | R4220003 |

| (min) | AVG | SD | AVG | SD | AVG | SD |
|---|---|---|---|---|---|---|
| 5 | 17.1 | 1.91 | 17.4 | 2.82 | 17.2 | 0.09 |
| 10 | 38.2 | 1.18 | 38.4 | 1.70 | 38.3 | 0.77 |
| 15 | 55.4 | 1.34 | 55.4 | 0.88 | 55.4 | 0.35 |
| 30 | 88.5 | 1.35 | 88.2 | 0.38 | 88.3 | 0.19 |
| 45 | 98.2 | 0.57 | 96.6 | 0.17 | 97.3 | 0.60 |
| 60 | 98.5 | 0.54 | 97.4 | 0.48 | 97.8 | 0.33 |

| | | | |
|---|---|---|---|
| f2 | NA | 99.4 | 99.8 |

TABLE 20
Summary of dissolution results of COMPOUND I tablets (50
mg) in acetate buffer pH 4.5 containing 1.0% SLS (n = 12)

| | COMPOUND I Tablets (Q ± SD, %) | | |
|---|---|---|---|
| Time | R4220001 | R4220002 | R4220003 |

| (min) | AVG | SD | AVG | SD | AVG | SD |
|---|---|---|---|---|---|---|
| 5 | 3.8 | 1.78 | 4.8 | 5.12 | 3.9 | 6.51 |
| 10 | 6.7 | 0.93 | 8.0 | 3.92 | 7.1 | 7.63 |
| 15 | 10.3 | 2.14 | 12.1 | 3.15 | 11.5 | 10.35 |
| 30 | 26.7 | 0.59 | 30.8 | 5.57 | 36.7 | 12.59 |
| 45 | 48.0 | 2.63 | 54.0 | 2.20 | 62.6 | 11.91 |
| 60 | 69.0 | 3.59 | 74.7 | 1.64 | 80.6 | 10.35 |
| 90 | 92.6 | 3.13 | 94.0 | 0.59 | 98.0 | 3.77 |
| 120 | 95.3 | 4.07 | 96.0 | 0.67 | 100.4 | 2.70 |

| | | | |
|---|---|---|---|
| f2 | NA | 71.1 | 54.0 |

TABLE 21
Summary of dissolution results of COMPOUND I tablets
(50 mg) in phosphate solution (pH 6.0) (n = 12)

| | COMPOUND I Tablets (Q ± SD, %) | | |
|---|---|---|---|
| time | R4220001 | R4220002 | R4220003 |

| (min) | AVG | SD | AVG | SD | AVG | SD |
|---|---|---|---|---|---|---|
| 5 | 14.0 | 4.98 | 14.2 | 4.65 | 13.6 | 2.94 |
| 10 | 34.5 | 2.98 | 35.4 | 3.46 | 34.6 | 0.09 |
| 15 | 50.2 | 2.53 | 51.4 | 2.95 | 50.6 | 0.05 |
| 30 | 81.1 | 1.80 | 82.1 | 0.91 | 81.0 | 0.24 |
| 45 | 90.5 | 0.46 | 90.6 | 0.32 | 90.9 | 0.07 |
| 60 | 91.7 | 0.53 | 91.1 | 0.57 | 91.2 | 0.08 |

| | | | |
|---|---|---|---|
| f2 | NA | 94.5 | 99.0 |

TABLE 22
Summary of dissolution results of COMPOUND
I tablets (50 mg) in pure water (n = 12)

| | COMPOUND I Tablets (Q ± SD, %) | | |
|---|---|---|---|
| Time | R4220001 | R4220002 | R4220003 |

| (min) | AVG | SD | AVG | SD | AVG | SD |
|---|---|---|---|---|---|---|
| 5 | 36.5 | 3.05 | 36.6 | 2.31 | 36.2 | 1.39 |
| 10 | 68.7 | 2.10 | 70.2 | 0.26 | 68.5 | 1.11 |
| 15 | 88.8 | 1.41 | 89.8 | 0.72 | 88.5 | 1.07 |
| 30 | 103.3 | 0.53 | 101.9 | 0.24 | 102.1 | 0.17 |
| 45 | 102.9 | 0.75 | 102.1 | 0.30 | 102.1 | 0.21 |
| 60 | 103.4 | 0.56 | 102.2 | 0.49 | 102.4 | 0.88 |

| | | | |
|---|---|---|---|
| f2 | NA | 92.0 | 99.2 |

TABLE 23
Summary of dissolution results of COMPOUND I tablets
(5 mg) in phosphate solution (pH 6.8) (n = 12)

| | COMPOUND I Tablets (Q ± SD, %) | | |
|---|---|---|---|
| time | R4220004 | R4220005 | R4220006 |

| (min) | AVG | SD | AVG | SD | AVG | SD |
|---|---|---|---|---|---|---|
| 5 | 12.6 | 5.44 | 9.5 | 21.60 | 12.2 | 2.51 |
| 10 | 33.2 | 2.70 | 31.6 | 0.44 | 33.2 | 3.93 |
| 15 | 50.7 | 0.51 | 48.8 | 0.39 | 51.0 | 3.18 |
| 30 | 77.1 | 1.49 | 75.3 | 1.09 | 78.4 | 3.75 |
| 45 | 87.3 | 1.68 | 84.4 | 1.11 | 86.9 | 3.58 |
| 60 | 90.6 | 2.21 | 87.6 | 0.01 | 89.7 | 3.71 |

| | | | |
|---|---|---|---|
| f2 | NA | 79.7 | 96.2 |

TABLE 24
Summary of dissolution results of COMPOUND I tablets (5
mg) in acetate buffer pH 4.5 containing 1.0% SLS (n = 12)

| | COMPOUND I Tablets (Q ± SD, %) | | |
|---|---|---|---|
| Time | R4220004 | R4220005 | R4220006 |

| (min) | AVG | SD | AVG | SD | AVG | SD |
|---|---|---|---|---|---|---|
| 5 | 10.3 | 9.10 | 9.3 | 2.93 | 10.3 | 3.55 |
| 10 | 19.6 | 12.60 | 17.8 | 0.01 | 20.1 | 1.05 |
| 15 | 29.4 | 14.02 | 27.2 | 2.13 | 30.3 | 0.48 |
| 30 | 54.8 | 12.60 | 52.5 | 2.20 | 57.8 | 0.14 |
| 45 | 71.1 | 9.96 | 71.0 | 1.96 | 77.2 | 0.86 |
| 60 | 82.7 | 7.93 | 82.2 | 3.28 | 88.1 | 0.40 |
| 90 | 94.1 | 2.81 | 93.7 | 2.59 | 96.1 | 0.84 |
| 120 | 99.6 | 0.58 | 99.0 | 2.57 | 99.2 | 0.47 |

| | | | |
|---|---|---|---|
| f2 | NA | 87.7 | 72.6 |

TABLE 25
Summary of dissolution results of COMPOUND I tablets
(5 mg) in phosphate solution (pH 6.0) (n = 12)

| | COMPOUND I Tablets (Q ± SD, %) | | |
|---|---|---|---|
| time | R4220004 | R4220005 | R4220006 |

| (min) | AVG | SD | AVG | SD | AVG | SD |
|---|---|---|---|---|---|---|
| 5 | 5.1 | 17.83 | 5.5 | 37.15 | 6.3 | 4.94 |
| 10 | 24.8 | 2.88 | 23.6 | 2.32 | 24.1 | 1.97 |
| 15 | 41.2 | 2.79 | 44.2 | 0.10 | 39.6 | 1.32 |
| 30 | 63.7 | 1.52 | 70.4 | 0.24 | 65.4 | 1.00 |
| 45 | 72.7 | 3.42 | 79.3 | 0.45 | 73.4 | 2.16 |
| 60 | 77.1 | 1.51 | 82.0 | 0.98 | 75.5 | 0.64 |

| | | | |
|---|---|---|---|
| f2 | NA | 66.7 | 89.1 |

TABLE 26
Summary of dissolution results of COMPOUND
I tablets (5 mg) in pure water (n = 12)

| | COMPOUND I Tablets (Q ± SD, %) | | |
|---|---|---|---|
| time | R4220004 | R4220005 | R4220006 |

| (min) | AVG | SD | AVG | SD | AVG | SD |
|---|---|---|---|---|---|---|
| 5 | 25.2 | 13.88 | 22.8 | 3.91 | 22.2 | 2.32 |
| 10 | 52.0 | 5.77 | 49.9 | 2.26 | 50.2 | 0.99 |
| 15 | 72.5 | 3.26 | 71.4 | 3.50 | 71.6 | 0.11 |
| 30 | 96.1 | 0.79 | 95.5 | 2.92 | 95.1 | 0.46 |
| 45 | 99.4 | 0.13 | 97.7 | 2.33 | 97.5 | 0.08 |
| 60 | 99.6 | 0.13 | 98.3 | 2.48 | 98.1 | 0.46 |

| | | | |
|---|---|---|---|
| f2 | NA | 85.1 | 83.6 |

### 2.4 Result

Compared dissolution profile in pH 1.0 and pH 4.5 with that in pH 6.8, the dissolution in pH 6.8 buffer is obviously better than other dissolution medium and meet the specification of COMPOUND I tablets (NLT 75% in 45 min). In addition, according to absorption contrast of COMPOUND I tablets in Beagle dogs, the dissolution condition in pH 6.8 buffer is significantly more discriminative for COMPOUND I tablets

In summary, pH 6.8 is the standard dissolution medium used in the dissolution test of COMPOUND I tablets.

### 3. Crystal Form

Pre-formulation studies showed that the crystal form of COMPOUND I tablets after the manufacturing process remained amorphous, which was exactly the same with COMPOUND I drug substance. Samples were planned to be taken at key time points of stability studies to continue to study the crystal forms of COMPOUND I in COMPOUND I tablets under long-term and accelerated conditions.

While various embodiments have been described above, it should be understood that such disclosures have been presented by way of example only and are not limiting. Thus, the breadth and scope of the subject compositions and methods should not be limited by any of the above-described exemplary embodiments, but should be defined only in accordance with the following claims and their equivalents.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

1. A pharmaceutical composition, comprising:
a) a compound shown in formula (I); 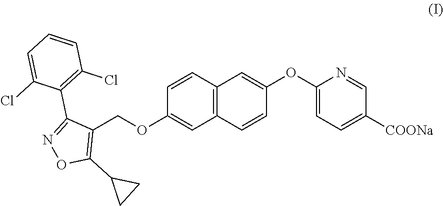
b) a solubilizing agent;
c) other pharmaceutically acceptable excipients.

2. The pharmaceutical composition of claim 1, comprising, in weight parts:
a) 1 part compound shown in formula (I);
b) 0.2 to 15 parts of a solubilizing agent;
c) 1 to 25 parts of other pharmaceutically acceptable excipients.

3. The pharmaceutical composition of claim 1, comprising, in weight parts:
a) 1 part compound shown in formula (I);
b) 0.2 to 15 parts of polyvinylpyrrolidone as solubilizing agent;
c) 1 to 25 parts of other pharmaceutically acceptable excipients.

4. The pharmaceutical composition of claim 1, comprising, on a weight-portion basis,
1 part of the compounds shown in formula (I);
1 to 10 parts of polyvinylpyrrolidone; and
1.7 to 20 parts of other pharmaceutically acceptable excipients.

5. The pharmaceutical composition of claim 1, wherein the other pharmaceutically acceptable excipients are selected from the group consisting of fillers, disintegrants, lubricants and glidants.

6. The pharmaceutical composition of claim 1, wherein the other pharmaceutically acceptable excipients comprise a filler selected from the group consisting of lactose, mannitol, silicified microcrystalline cellulose, dibasic calcium phosphate, microcrystalline cellulose, starch and pregelatinized starch.

7. The pharmaceutical composition of claim 1, wherein the other pharmaceutically acceptable excipients comprise a disintegrant selected from the group consisting of cross-linked polyvidone, croscarmellose sodium and croscarmellose sodium.

8. The pharmaceutical composition of claim 1, wherein the other pharmaceutically acceptable excipients comprise a lubricant selected from the group consisting of sodium stearyl fumarate, magnesium stearate and talc.

9. The pharmaceutical composition of claim 1, wherein the other pharmaceutically acceptable excipients comprise an auxiliary agent selected from the group consisting of colloidal silicon dioxide, magnesium aluminum silicate and polyethylene glycol.

10. The pharmaceutical composition of claim 1, wherein the compound of formula (I) is present in an amorphous form.

11. The pharmaceutical composition of claim 1, wherein the pharmaceutical composition is in a solid dosage form.

12. The pharmaceutical composition of claim 1, comprising, in weight parts:
1 part of the compound shown in (I);
1 part of polyvinylpyrrolidone;
0.8 part of mannitol;
0.6 part silicified microcrystalline cellulose;
0.4 part of cross-linked polyvidone; and
0.2 part of sodium stearyl fumarate.

13. The pharmaceutical composition of claim 1, comprising, in weight parts:
1 part of the compound shown in (I);
10 part of polyvinylpyrrolidone;
8 part of mannitol;
6 part silicified microcrystalline cellulose;
4 part of cross-linked polyvidone; and
2 part of sodium stearyl fumarate.

14. A method of making a pharmaceutical composition, comprising the steps of:
mixing a compound of formula (I) 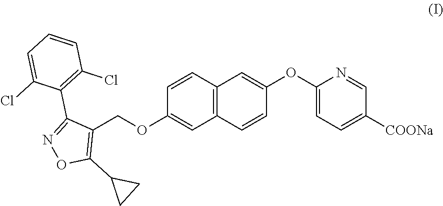
with one or more solubilizing agents and/or one or more other pharmaceutically acceptable excipients to form a first mixture;
granulating the first mixture to form a granulation product; and
producing the pharmaceutical composition in a dosage form with the granulation product.

15. The method of claim 14, wherein the producing step comprises:
blending the granulation product with one or more additional pharmaceutically acceptable carrier to form a tableting mixture; and
compressing the tableting mixture into tablets.

16. The method of claim 14, wherein one or more of the compound of formula (I), the one or more solubilizing agents, and/or the one or more other pharmaceutically acceptable excipients are grinded and/or sieved before the mixing step.

17. The method of claim 16, wherein the compound of formula (I) is sieved with a 30-mesh sieve before the mixing step, and wherein the one or more solubilizing agents and/or the one or more other pharmaceutically acceptable excipients are sieved with a 40-mesh sieve before the mixing step.

18. The method of claim 14, wherein one or more of the compound of formula (I), the one or more solubilizing agents and/or the one or more other pharmaceutically acceptable excipients are grinded by mechanical grinding and/or airflow grinding.

19. A pharmaceutical composition produced by the method of claim 14.

20. A method of treating nonalcoholic steatohepatitis in a subject, comprising the step of:
administering to a subject in need of such treatment, an effective amount of the pharmaceutical composition of claim 1.
